(19) United States
(12) Patent Application Publication
(10) Pub. No.: US 2008/0280945 A1
(43) Pub. Date: Nov. 13, 2008

(54) Crystalline forms of an HIV integrase inhibitor

(76) Inventors: Sachin Lohani, Annandale, NJ (US); Zhihui Peng, Edison, NJ (US); Arlene E. McKeown, Summit, NJ (US)

Correspondence Address: MERCK AND CO., INC, P O BOX 2000, RAHWAY, NJ, 07065-0907, US

(21) Appl. No.: 12/151,744

(22) Filed: May 8, 2008

Related U.S. Application Data
(60) Provisional application No. 60/928,292, filed on May 9, 2007.

(51) Int. Cl. A61K 31/4375; C07D 471/14; A61P 31/18

(52) U.S. Cl. 514/293; 540/460

(57) ABSTRACT

Crystalline forms of a hexahydro-diazocinonaphthyridine trione compound are disclosed. The compound and its crystalline forms thereof are HIV integrase inhibitors useful for the prophylaxis or treatment of HIV infection or for the prophylaxis, treatment or delay in the onset or progression of AIDS.

### CROSS-REFERENCE TO RELATED APPLICATIONS

This application claims the benefit of U.S. Provisional Application No. 60/928,292, filed May 9, 2007, the disclosure of which is incorporated by reference herein in its entirety.

### FIELD OF THE INVENTION

The present invention is directed to crystalline forms of a hexahydro-diazocinonaphthyridine trione HIV integrase inhibitor identified below as Isomer M, methods of preparing the crystalline forms, pharmaceutical compositions containing the crystalline forms, and use of the forms in the treatment or prophylaxis of HIV infection or in the treatment, prophylaxis, or delay in the onset or progression of AIDS.

### BACKGROUND OF THE INVENTION

The HIV retrovirus, particularly the strains known as type-1 (HIV-1) virus and type-2 (HIV-2) virus, is the causative agent for AIDS. The HIV-1 retrovirus primarily uses the CD4 receptor (a 58 kDa transmembrane protein) to gain entry into cells, through high-affinity interactions between the viral envelope glycoprotein (gp120) and a specific region of the CD4 molecule found in T-lymphocytes and CD4 (+) T-helper cells (Lasky L. A. et al., Cell 1987, 50: 975-985). HIV infection is characterized by an asymptomatic period immediately following infection that is devoid of clinical manifestations in the patient. Progressive HIV-induced destruction of the immune system then leads to increased susceptibility to opportunistic infections, which eventually produces a syndrome called ARC (AIDS-related complex) characterized by symptoms such as persistent generalized lymphadenopathy, fever, and weight loss, followed itself by full blown AIDS.

After entry of the retrovirus into a cell, viral RNA is converted into DNA, which is then integrated into the host cell DNA. Integration of viral DNA is an essential step in the viral life cycle. Integration is believed to be mediated by integrase, a 32 kDa enzyme, in three steps: assembly of a stable nucleoprotein complex with viral DNA sequences; cleavage of two nucleotides from the 3′ termini of the linear proviral DNA; and covalent joining of the recessed 3′ OH termini of the proviral DNA at a staggered cut made at the host target site. The fourth step in the process, repair synthesis of the resultant gap, may be accomplished by cellular enzymes.

The compound (4R)-11-(3-chloro-4-fluorobenzyl)-4,9-dihydroxy-2,5,5-trimethyl-3,4,5,6,12,13-hexahydro-2H[1,4]diazocino[2,1-a]-2,6-naphthyridine-1,8,10(11H)-trione is referred to herein as “Compound A” and has the following structure:

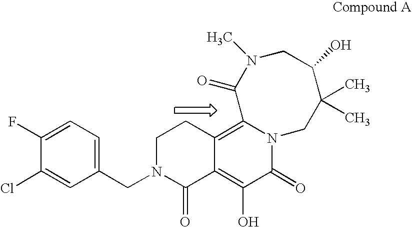

Compound A has two isomers as a result of atropisomerism. Atropisomerism is observed when the otherwise free rotation about a bond is sufficiently restricted (e.g., by the presence of a bulky substituent) to result in rotational enantiomers called atropisomers whose interconversion is sufficiently slow to allow for their separation and characterization. See, e.g., J. March, Advanced Organic Chemistry, 4th Edition, John Wiley & Sons, 1992, pp. 101-102; and Ahmed et al., Tetrahedron 1998, 13277 for further description of atropisomerism. The foregoing compound has sufficient hindrance to rotation along the bond indicated with the arrow to permit separation of the enantiomers (using, e.g., column chromatography on a chiral stationary phase). Using Helical nomenclature for assigning atropisomers (see Prelog et al., Angew. Chem. Int. Ed. Engl. 1992, 21: 567-583) the atropisomers of the foregoing compound are M-(4R)-1′-(3-chloro-4-fluorobenzyl)-4,9-dihydroxy-2,5,5-trimethyl-3,4,5,6,12,13-hexahydro-2H[1,4]diazocino[2,1-a]-2,6-naphthyridine-1,8,10(11H)-trione (alternatively referred to herein as “Isomer M”) and P-(4R)-11-(3-chloro-4-fluorobenzyl)-4,9-dihydroxy-2,5,5-trimethyl-3,4,5,6,12,13-hexahydro-2H[1,4]diazocino[2,1-a]-2,6-naphthyridine-1,8,10(11H)-trione (“Isomer P”). Isomer M and Isomer P are both HIV integrase inhibitors.

Example 38 of WO 2006/121831 discloses Compound A. Example 38 further discloses the preparation of Compound A via (3R)-3-(benzyloxy)-4,4-dimethyldihydrofuran-2(3H)-one which is prepared from D(−)-pantolactone. It was subsequently discovered that the preparative route disclosed in Example 38 provides a racemic, amorphous mixture of the 4R and 4S enantiomers. It was determined that the installation of the benzyl protective group on the hydroxyl group of optically pure D-pantolactone employed in Example 38 results in racemization of the chiral center. It was also discovered, as shown in Example 1, steps 10 et seq. below, that installation of a 2-tetrahydropyranyl protective group does not lead to racemization, but instead results in the 4R isomer, from which Isomer M can be obtained in an amorphous form.

In pharmaceutical applications, the use of a crystalline form of a drug substance is typically preferred over an amorphous form thereof. The amorphous state is typically less stable thermodynamically compared to a crystalline state. Consequently, amorphous materials are typically more hygroscopic and susceptible to physical and chemical change over time than their crystalline counterparts. In order to minimize such change, amorphous materials often require special handling, which can include preparation, formulation and/or storage under carefully controlled conditions (e.g., low temperatures and low humidity levels). It is also more difficult to control the impurity content and the material properties (e.g., particle size and morphology) of an amorphous drug substance. Accordingly, the use of a crystalline material as the active ingredient in a drug product can result in an improved product characterized by having a lower impurity content and more robust and predictable chemical and physical behavior, thereby reducing or eliminating the need for stringent handling procedures.

### SUMMARY OF THE INVENTION

The present invention is directed to crystalline forms of Isomer M. The present invention also includes methods of preparing a crystalline form of Isomer M, pharmaceutical compositions containing a crystalline form of Isomer M, methods of using a crystalline form of Isomer M (either alone or in combination with another HIV integrase inhibitor) for inhibition of HIV integrase (e.g., HIV-1 integrase), and methods of using a crystalline form of Isomer M (either alone or in combination other HIV/AIDS antivirals, anti-infectives, immunomodulators, antibiotics or vaccines) for prophylaxis or treatment of HIV infection, or for the prophylaxis, treatment or delay in the onset or progression of AIDS.

Embodiments, aspects and features of the present invention are either further described in or will be apparent from the ensuing description, examples, and appended claims.

The crystalline forms of Isomer M of the present invention are a crystalline ethanolate, a crystalline hydrate, and a crystalline anhydrate. All three of these forms exhibit significantly better thermal and moisture stability relative to the amorphous form, with the crystalline anhydrate exhibiting the most stability.

### BRIEF DESCRIPTION OF THE DRAWINGS

FIG. 1 is the X-ray powder diffraction pattern for the amorphous form of Isomer M prepared in accordance with the method described in Example 1.

FIG. 2 is the X-ray powder diffraction pattern for the crystalline ethanolate in Example 2.

FIG. 3 is the DSC curve for the crystalline ethanolate in Example 2.

FIG. 4 is a plot of the thermogravimetric analysis for the crystalline ethanolate in Example 2.

FIG. 5 is the C-13 CPMAS spectrum for the crystalline ethanolate in Example 2.

FIG. 6 is the X-ray powder diffraction pattern for the crystalline hydrate in Example 3.

FIG. 7 is the DSC curve for the crystalline hydrate in Example 3.

FIG. 8 is a plot of the thermogravimetric analysis for the crystalline hydrate in Example 3.

FIG. 9 is the C-13 CPMAS spectrum for the crystalline hydrate in Example 3.

FIG. 10 is the F-19 CPMAS spectrum for the crystalline hydrate in Example 3.

FIG. 11 is the X-ray powder diffraction pattern for the crystalline anhydrate in Example 4.

FIG. 12 is the DSC curve for the crystalline anhydrate in Example 4.

FIG. 13 is a plot of the thermogravimetric analysis for the crystalline anhydrate in Example 4.

FIG. 14 is the C-13 CPMAS spectrum for the crystalline anhydrate in Example 4.

FIG. 15 is the F-19 CPMAS spectrum for the crystalline anhydrate in Example 4.

### DETAILED DESCRIPTION OF THE INVENTION

The present invention includes crystalline forms of Isomer M, pharmaceutical compositions containing the crystalline forms, and methods of making and using the crystalline forms. The crystalline forms of Isomer M and pharmaceutical compositions containing the crystalline forms are useful for inhibiting HIV integrase (e.g., HIV-1 integrase), prophylaxis of infection by HIV, treating infection by HIV, delaying the onset or progression of AIDS, prophylaxis of AIDS, and treating AIDS, in adults, children or infants. Treating HIV infection is defined as including, but not limited to, treatment of a wide range of states of HIV infection: AIDS, ARC, both symptomatic and asymptomatic, and actual or potential exposure to HIV. For the purposes of the prophylaxis of AIDS, the treatment of AIDS or a delay in the onset or progression of AIDS, AIDS includes either or both AIDS and ARC. For example, the crystalline forms of Isomer M and pharmaceutical compositions thereof are useful in treating infection by HIV after suspected past exposure to HIV by, e.g., blood transfusion, exchange of body fluids, bites, accidental needle stick, or exposure to patient blood during surgery. The crystalline forms can also be used in “salvage” therapy; i.e., they can be used to treat HIV infection or AIDS in HIV-positive subjects whose viral load achieved undetectable levels via conventional therapies (e.g., therapies employing known protease inhibitors in combination with one or more known reverse transcriptase inhibitors), and then rebounded due to the emergence of HIV mutants resistant to the known inhibitors.

Isomer M is an inhibitor of HIV integrase, in particular HIV-1 integrase. Isomer M has been tested in an integrase inhibition assay in which strand transfer is catalyzed by recombinant integrase, and has been found to be a potent inhibitor. More particularly, Isomer M has been tested in the strand transfer assay described in Example 193 of WO 02/30930 for recombinant integrase and found to have an IC₅₀ value of 13.5±7.0 (n=4) μM.

Isomer M has also been found to be active in an assay for the inhibition of acute HIV infection of T-lymphoid cells conducted in accordance with Vacca et al., Proc. Natl. Acad. Sci. USA 1994, 91: 4096-4100. More particularly, Isomer M has been found to have an IC₉₅ value of 15.6±0 (n=6) 1M in the presence of 10% FBS and a value of 51.7±16 (n=6) μM in the presence of 50% human serum.

A first embodiment of the present invention (alternatively referred to herein as “Embodiment E1”) is a crystalline ethanolate of Isomer M, which is characterized by an X-ray powder diffraction pattern obtained using copper K_{α} radiation (i.e., the radiation source is a combination of Cu K_{α1} and K_{α2} radiation) which comprises 2Θ values (i.e., reflections at 2Θ values) in degrees of about 17.8, 19.9, 21.0, and 21.8. In this embodiment and analogous embodiments which follow the term “about” is understood to modify each of the 2Θ values; i.e., the expression “about 17.8, 19.9, 21.0, and 21.8” is short-hand for “about 17.8, about 19.9, about 21.0, and about 21.8”.

A second embodiment of the present invention (Embodiment E2) is a crystalline ethanolate of Isomer M, which is characterized by an X-ray powder diffraction pattern obtained using copper K_{α} radiation which comprises 2Θ values in degrees of about 17.8, 19.9, 21.0, 21.8, 13.2, 13.6, 19.1, and 23.0.

A third embodiment of the present invention (Embodiment E3) is a crystalline ethanolate of Isomer M, which is characterized by an X-ray powder diffraction pattern obtained using copper K_{α} radiation which comprises 2Θ values in degrees of about 17.8, 19.9, 21.0, 21.8, 13.2, 13.6, 19.1, 23.0, 20.1, 22.2, 26.0, and 28.2.

A fourth embodiment of the present invention (Embodiment E4) is a crystalline ethanolate of Isomer M as defined in any one of Embodiments E1 to E3, which is further characterized by a carbon-13 CPMAS spectrum comprising the chemical shifts in Table 3 below.

A fifth embodiment of the present invention (Embodiment E5) is a crystalline ethanolate of Isomer M as defined in any one of Embodiments E1 to E4, which is further characterized by a differential scanning calorimetry curve, obtained at a heating rate of 10° C./minute in an open aluminum pan under nitrogen, exhibiting an endotherm with an onset temperature of about 138° C. and a peak temperature of about 142° C.

A sixth embodiment of the present invention (Embodiment E6) is a crystalline hydrate characterized by an X-ray powder diffraction pattern obtained using copper K_{α} radiation which comprises 2Θ values in degrees of about 13.5, 14.1, 17.8, and 19.9.

A seventh embodiment of the present invention (Embodiment E7) is a crystalline hydrate characterized by an X-ray powder diffraction pattern obtained using copper K_{α} radiation which comprises 2Θ values in degrees of about 13.5, 14.1, 17.8, 19.9, 10.3, 18.8, 24.3, and 33.5.

An eighth embodiment of the present invention (Embodiment E8) is a crystalline hydrate characterized by an X-ray powder diffraction pattern obtained using copper K_{α} radiation which comprises 2Θ values in degrees of about 13.5, 14.1, 17.8, 19.9, 10.3, 18.8, 24.3, 33.5, 17.4, 26.4, 27.2, and 29.8.

A ninth embodiment of the present invention (Embodiment E9) is a crystalline hydrate of Isomer M as defined in any one of Embodiments E6 to E8, which is further characterized by a carbon-13 CPMAS spectrum comprising the chemical shifts in Table 5 below.

A tenth embodiment of the present invention (Embodiment E10) is a crystalline hydrate of Isomer M as defined in any one of Embodiments E6 to E9, which is further characterized by a differential scanning calorimetry curve, obtained at a heating rate of 10° C./minute in an open aluminum pan under nitrogen, exhibiting a first endotherm with an onset temperature of about 72° C. and a peak temperature of about 109° C. and a second endotherm with an onset temperature of about 243° C. and a peak temperature of about 244° C.

An eleventh embodiment of the present invention (Embodiment E11) is a crystalline hydrate of Isomer M as defined in any one of Embodiments E6 to E10, which is further characterized by a fluorine-19 CPMAS spectrum having a chemical shift of about −118.4 ppm.

A twelfth embodiment of the present invention (Embodiment E12) is a crystalline anhydrate characterized by an X-ray powder diffraction pattern obtained using copper K_{α} radiation which comprises 2Θ values in degrees of about 10.0, 16.0, 20.2, and 23.8.

A thirteenth embodiment of the present invention (Embodiment E13) is a crystalline anhydrate characterized by an X-ray powder diffraction pattern obtained using copper K_{α} radiation which comprises 2Θ values in degrees of about 10.0, 16.0, 20.2, 23.8, 11.8, 15.8, 21.1, and 24.7.

A fourteenth embodiment of the present invention (Embodiment E14) is a crystalline anhydrate characterized by an X-ray powder diffraction pattern obtained using copper K_{α} radiation which comprises 2Θ values in degrees of about 10.0, 16.0, 20.2, 23.8, 11.8, 15.8, 21.1, 24.7, 15.4, 17.1, 25.7, and 29.3.

A fifteenth embodiment of the present invention (Embodiment E15) is a crystalline anhydrate of Isomer M as defined in any one of Embodiments E12 to E14, which is further characterized by a carbon-13 CPMAS spectrum comprising the chemical shifts in Table 7 below.

A sixteenth embodiment of the present invention (Embodiment E16) is a crystalline anhydrate of Isomer M as defined in any one of Embodiments E12 to E15, which is further characterized by a differential scanning calorimetry curve, obtained at a heating rate of 10° C./minute in a covered aluminum pan under nitrogen, exhibiting an endotherm with an onset temperature of about 242° C. and a peak temperature of about 243° C.

A seventeenth embodiment of the present invention (Embodiment E17) is a crystalline anhydrate of Isomer M as defined in any one of Embodiments E12 to E16, which is further characterized by a fluorine-19 CPMAS spectrum having a chemical shift of about −115.6 ppm.

The crystalline forms of Isomer M as set forth in the foregoing embodiments can alternatively be described in terms of the crystallographic d-spacings corresponding to the 2Θ reflections. The corresponding d-spacings are listed in Examples 2 to 4 below.

An eighteenth embodiment of the present invention (Embodiment E18) is crystalline Isomer M as defined in any of the foregoing embodiments, wherein the crystal form is substantially pure. As used herein “substantially pure” means that the crystalline form contains Isomer M (e.g., in a product isolated from a process for obtaining the crystalline form of Isomer M) in an amount of at least about 90 wt. % (e.g., from about 95 wt. % to 100 wt. %), preferably at least about 95 wt. % (e.g., from about 98 wt. % to 100 wt. %), more preferably at least about 99 wt. %, and most preferably 100 wt. %. The relative amount of Isomer M in the crystalline form can be determined using a suitable standard method of analysis such as using a titration method or HPLC in conjunction with chiral chromatography. If more than one method of analysis is employed and the methods provide experimentally significant differences in the level of purity determined, then the method providing the highest purity level governs. A crystalline form of 100% purity can alternatively be described as one which is free of a detectable amount of Isomer P and/or other impurities as determined by a suitable method of analysis.

Other embodiments of the present invention include the following:

(a) A pharmaceutical composition comprising an effective amount of a crystalline form of Isomer M and a pharmaceutically acceptable carrier.

(b) The pharmaceutical composition of (a), wherein the composition is a solid dosage form suitable for oral administration (e.g., a tablet or a capsule).

(c) The pharmaceutical composition of (a) or (b), further comprising an effective amount of an anti-HIV agent selected from the group consisting of HIV antiviral agents, immunomodulators, and anti-infective agents.

(d) The pharmaceutical composition of (c), wherein the anti-HIV agent is an antiviral selected from the group consisting of HIV protease inhibitors, HIV integrase inhibitors other than Compound A, nucleoside HIV reverse transcriptase inhibitors, non-nucleoside HIV reverse transcriptase inhibitors, and HIV fusion inhibitors.

(e) A pharmaceutical combination which is (i) a crystalline form of Isomer M and (ii) an anti-HIV agent selected from the group consisting of HIV antiviral agents, immunomodulators, and anti-infective agents; wherein the crystalline form of Isomer M and the anti-HIV agent are each employed in an amount that renders the combination effective for inhibition of HIV integrase, for treatment or prophylaxis of infection by HIV, or for treatment, prophylaxis of, or delay in the onset or progression of AIDS.

(f) The combination of (e), wherein the anti-HIV agent is an antiviral selected from the group consisting of HIV protease inhibitors, HIV integrase inhibitors other than Compound A, nucleoside HIV reverse transcriptase inhibitors, non-nucleoside HIV reverse transcriptase inhibitors, and HIV fusion inhibitors.

(g) A method for the inhibition of HIV integrase in a subject in need thereof which comprises administering to the subject an effective amount of a crystalline form of Isomer M.

(h) A method of the prophylaxis or treatment of infection by HIV (e.g., HIV-1) in a subject in need thereof which comprises administering to the subject an effective amount of a crystalline form of Isomer M.

(i) The method of (h), wherein the crystalline form of Isomer M is administered in combination with an effective amount of at least one other HIV antiviral selected from the group consisting of HIV protease inhibitors, HIV integrase inhibitors other than Compound A, nucleoside HIV reverse transcriptase inhibitors, non-nucleoside HIV reverse transcriptase inhibitors, and HIV fusion inhibitors.

(j) A method for the prophylaxis, treatment or delay in the onset or progression of AIDS in a subject in need thereof which comprises administering to the subject an effective amount of a crystalline form of Isomer M.

(k) The method of (j), wherein the compound is administered in combination with an effective amount of at least one other HIV antiviral selected from the group consisting of HIV protease inhibitors, HIV integrase inhibitors other than Compound A, nucleoside HIV reverse transcriptase inhibitors, non-nucleoside HIV reverse transcriptase inhibitors, and HIV fusion inhibitors.

(l) A method for the inhibition of HIV integrase in a subject in need thereof which comprises administering to the subject the pharmaceutical composition of (a), (b), (c) or (d) or the combination of (e) or (f).

(m) A method for the prophylaxis or treatment of infection by HIV (e.g., HIV-1) in a subject in need thereof which comprises administering to the subject the pharmaceutical composition of (a), (b), (c) or (d) or the combination of (e) or (f).

(n) A method for the prophylaxis, treatment, or delay in the onset or progression of AIDS in a subject in need thereof which comprises administering to the subject the pharmaceutical composition of (a), (b), (c) or (d) or the combination of (e) or (f).

The present invention also includes a crystalline form of Isomer M (i) for use in, (ii) for use as a medicament for, or (iii) for use in the preparation of a medicament for: (a) therapy (e.g., of the human body), (b) medicine, (c) inhibition of HIV reverse transcriptase, (d) treatment or prophylaxis of infection by HIV, or (e) treatment, prophylaxis of, or delay in the onset or progression of AIDS. In these uses, the crystalline forms of the present invention can optionally be employed in combination with one or more anti-HIV agents selected from HIV antiviral agents, anti-infective agents, and immunomodulators.

Additional embodiments of the invention include the pharmaceutical compositions, combinations and methods set forth in (a)-(n) above and the uses (i) (a)-(e) through (iii) (a)-(e) set forth in the preceding paragraph, wherein the crystalline form of Isomer M of the present invention employed therein is a crystalline form as set forth in one of the embodiments E1 to E17 set forth above.

Additional embodiments of the present invention include each of the pharmaceutical compositions, combinations, methods and uses set forth in the preceding paragraphs, wherein the crystalline form of Isomer M employed therein is substantially pure. With respect to a pharmaceutical composition comprising a crystalline form of Isomer M and a pharmaceutically acceptable carrier and optionally one or more excipients, it is understood that the term “substantially pure” is in reference to the crystalline form of Isomer M per se.

Still additional embodiments of the present invention include the pharmaceutical compositions, combinations and methods set forth in (a)-(n) above and the uses (i) (a)-(e) through (iii) (a)-(e) set forth above, wherein the HIV of interest is HIV-1. Thus, for example, in the pharmaceutical composition (d), the crystalline form of Isomer M is employed in an amount effective against HIV-1 and the anti-HIV agent is an HIV-1 antiviral selected from the group consisting of HIV-1 protease inhibitors, HIV-1 integrase inhibitors other than Compound A, nucleoside HIV-1 reverse transcriptase inhibitors, non-nucleoside HIV-1 reverse transcriptase inhibitors, and HIV-1 fusion inhibitors.

The term “administration” and variants thereof (e.g., “administered” or “administering”) in reference to a crystalline form of Isomer M mean providing the crystal form to the individual in need of inhibition, treatment or prophylaxis. When a crystalline form of Isomer M is provided in combination with one or more other active agents (e.g., anti-HIV agents such as antiviral agents useful for the treatment or prophylaxis of HIV infection or AIDS), “administration” and its variants are each understood to include provision of the compound and other agents at the same time (separately or together) or at different times.

As used herein, the term “composition” is intended to encompass a product comprising the specified ingredients, as well as any product which results, directly or indirectly, from combining the specified ingredients.

By “pharmaceutically acceptable” is meant that the ingredients of the pharmaceutical composition must be compatible with each other and not deleterious to the recipient thereof.

The term “subject” as used herein refers to an animal, preferably a mammal, most preferably a human, who has been the object of treatment, observation or experiment.

The term “effective amount” as used herein means that amount of active compound or pharmaceutical agent that elicits the biological or medicinal response in a tissue, system, animal or human that is being sought by a researcher, veterinarian, medical doctor or other clinician. The effective amount can be a “therapeutically effective amount” for the alleviation of the symptoms of the disease or condition being treated. The effective amount can also be a “prophylactically effective amount” for prophylaxis of the symptoms of the disease or condition being prevented. The term also includes herein the amount of active compound sufficient to inhibit HIV integrase and thereby elicit the response being sought (i.e., an “inhibition effective amount”).

The term “anti-HIV agent” is any agent which is directly or indirectly effective in the inhibition of HIV integrase or another enzyme required for HIV replication or infection, in the treatment or prophylaxis of HIV infection, and/or in the treatment, prophylaxis or delay in the onset or progression of AIDS. It is understood that an anti-HIV agent is effective in treating, preventing, or delaying the onset or progression of HIV infection or AIDS and/or diseases or conditions arising therefrom or associated therewith.

In the methods encompassed by the present invention (i.e., inhibiting HIV integrase, treating or prophylaxis of HIV infection or treating, prophylaxis of, or delaying the onset or progression of AIDS), the crystalline form of Isomer M can be administered by any means that produces contact of the active agent with the agent's site of action. The crystalline form can be administered by any conventional means available for use in conjunction with pharmaceuticals, either as individual therapeutic agents or in a combination of therapeutic agents. The crystal form of Isomer M can be administered alone, but is typically administered with a pharmaceutical carrier selected on the basis of the chosen route of administration and standard pharmaceutical practice. The crystalline form of Isomer M can, for example, be administered orally, parenterally (including subcutaneous injections, intravenous, intramuscular, intrasternal injection or infusion techniques), by inhalation spray, or rectally, in the form of a unit dosage of a pharmaceutical composition containing an effective amount of the crystal form and conventional non-toxic pharmaceutically acceptable carriers, adjuvants and vehicles. Liquid preparations suitable for oral administration (e.g., suspensions, syrups, elixirs and the like) can be prepared according to techniques known in the art and can employ any of the usual media such as water, glycols, oils, alcohols and the like. Solid preparations suitable for oral administration (e.g., powders, pills, capsules and tablets) can be prepared according to techniques known in the art and can employ such solid excipients as starches, sugars, kaolin, lubricants, binders, disintegrating agents and the like. Parenteral compositions can be prepared according to techniques known in the art and typically employ sterile water as a carrier and optionally other ingredients, such as a solubility aid. Injectable solutions can be prepared according to methods known in the art wherein the carrier comprises a saline solution, a glucose solution or a solution containing a mixture of saline and glucose. Further description of methods suitable for use in preparing pharmaceutical compositions for use in the present invention and of ingredients suitable for use in said compositions is provided in Remington's Pharmaceutical Sciences, 18^{th} edition, edited by A. R. Gennaro, Mack Publishing Co., 1990 and in Remington—The Science and Practice of Pharmacy, 21st edition, Lippincott Williams & Wilkins, 2005. The preferred method of administration of a crystalline form of Isomer M is oral administration in a solid dosage form, preferably in a capsule and more preferably in a tablet.

The crystalline forms of Isomer M of this invention can be administered orally in a dosage range of about 0.01 to about 100 mg/kg of mammal (e.g., human) body weight per day in a single dose or in divided doses. One preferred dosage range is about 0.05 to about 50 mg/kg body weight per day orally in a single dose or in divided doses. For oral administration, the compositions can be provided in the form of tablets or capsules containing about 1.0 to about 500 milligrams of the active ingredient, particularly 1, 5, 10, 15, 20, 25, 50, 75, 100, 150, 200, 250, 300, 400, and 500 milligrams of the active ingredient, for the symptomatic adjustment of the dosage to the patient to be treated. The specific dose level and frequency of dosage for any particular patient can be varied and will depend upon a variety of factors including the age, body weight, general health, sex, diet, mode and time of administration, rate of excretion, drug combination, the severity of the particular condition, and the host undergoing therapy.

In one embodiment, crystalline forms of Isomer M can be administered to adult humans in capsules or tablets in an amount in a range of from about 5 mg to about 800 mg once or twice/day. A suitable formulation consists of:

| | Concentration |
|---|---|
| Component | (wt. %) |
| Isomer M | 5-25 |
| Na lauryl sulfate | 2 |
| lactose monhydrate | 34-44 |
| microcrystalline cellulose | 34-44 |
| croscarmellose Na | 3 |
| Na stearyl fumarate | 2 |

The formulation can be prepared by blending the crystalline from of Isomer M, sodium lauryl sulfate, lactose, microcrystalline cellulose, and croscarmellose sodium; adding a portion of the sodium stearyl fumarate (e.g., half) and blending further; roller compacting the blend to form compacted ribbons; milling the ribbons into granules; and lubricating the granules with the balance of the sodium stearyl fumarate; and either compacting the lubricated granules into tablets or encapsulating the lubricated granules in hard gelatin capsules containing, for example, 5 mg, 25 mg, and 100 mg of active ingredient.

As noted above, the present invention is also directed to use of a crystalline form of Isomer M with one or more anti-HIV agents useful in the treatment of HIV infection or AIDS. For example, a crystalline form of Isomer M of this invention may be effectively administered, whether at periods of pre-exposure and/or post-exposure, in combination with effective amounts of one or more HIV antivirals, immunomodulators, antiinfectives, or vaccines useful for treating HIV infection or AIDS, such as those disclosed in Table 1 of WO 01/38332 or in the Table in WO 02/30930. Suitable HIV antivirals for use in combination with a crystalline form of Isomer M of the present invention include, for example, those listed in Table 1 as follows:

TABLE 1

| Name | Type |
|---|---|
| abacavir, ABC, Ziagen ® | nRTI |
| abacavir + lamivudine, Epzicom ® | nRTI |
| abacavir + lamivudine + zidovudine, Trizivir ® | nRTI |
| amprenavir, Agenerase ® | PI |
| atazanavir, Reyataz ® | PI |
| AZT, zidovudine, azidothymidine, Retrovir ® | nRTI |
| capravirine | nnRTI |
| darunavir, Prezista ® | PI |
| ddC, zalcitabine, dideoxycytidine, Hivid ® | nRTI |
| ddI, didanosine, dideoxyinosine, Videx ® | nRTI |
| ddI (enteric coated), Videx EC ® | nRTI |
| delavirdine, DLV, Rescriptor ® | nnRTI |
| efavirenz, EFV, Sustiva ®, Stocrin ® | nnRTI |
| efavirenz + emtricitabine + tenofovir DF, Atripla ® | nnRTI + nRTI |
| emtricitabine, FTC, Emtriva ® | nRTI |
| emtricitabine + tenofovir DF, Truvada ® | nRTI |
| emvirine, Coactinon ® | nnRTI |
| enfuvirtide, Fuzeon ® | FI |
| enteric coated didanosine, Videx EC ® | nRTI |
| etravirine, TMC-125 | nnRTI |
| fosamprenavir calcium, Lexiva ® | PI |
| indinavir, Crixivan ® | PI |
| lamivudine, 3TC, Epivir ® | nRTI |
| lamivudine + zidovudine, Combivir ® | nRTI |
| lopinavir | PI |
| lopinavir + ritonavir, Kaletra ® | PI |
| maraviroc, Selzentry ® | EI |
| nelfinavir, Viracept ® | PI |
| nevirapine, NVP, Viramune ® | nnRTI |
| PPL-100 (also known as PL-462) (Ambrilia) | PI |
| raltegravir, MK-0518, Isentress ™ | InI |
| ritonavir, Norvir ® | PI |
| saquinavir, Invirase ®, Fortovase ® | PI |
| stavudine, d4T, didehydrodeoxythymidine, Zerit ® | nRTI |
| tenofovir DF (DF = disoproxil fumarate), TDF, | nRTI |
| Viread ® | |
| tipranavir, Aptivus ® | PI |

EI = entry inhibitor;
FI = fusion inhibitor;
InI = integrase inhibitor;
PI = protease inhibitor;
nRTI = nucleoside reverse transcriptase inhibitor;
nnRTI = non-nucleoside reverse transcriptase inhibitor.
Some of the drugs listed in the table are used in a salt form; e.g., abacavir sulfate, indinavir sulfate, atazanavir sulfate, nelfinavir mesylate.

The scope of combinations of a crystalline form of Isomer M of this invention with HIV antivirals, immunomodulators, anti-infectives or vaccines is not limited to the foregoing substances or to the lists in the above-referenced Tables in WO 01/38332 and WO 02/30930, but includes in principle any combination with any pharmaceutical composition useful for the treatment or prophylaxis of HIV infection or AIDS. The HIV antivirals and other agents will typically be employed in these combinations in their conventional dosage ranges and regimens as reported in the art, including, for example, the dosages described in the Physicians' Desk Reference, 58^{th} edition, Thomson PDR, 2004, or the 59^{th} edition thereof, 2005. The dosage ranges for a crystalline form of Isomer M of the present invention in these combinations are the same as those set forth above. It is understood that pharmaceutically acceptable salts of the other agents (e.g., indinavir sulfate) can be used as well.

The present invention also includes methods for preparing crystalline forms of Isomer M. More particularly, the present invention includes a first process (referred to herein as “Process P1”), which is a process for preparing a crystalline ethanolate of Isomer M as defined and described above, which comprises:

(A) dissolving Isomer M in methylene chloride;

(B) switching the solvent to ethanol to provide a slurry of the crystalline ethanolate;

(C) optionally ageing the slurry; and

(D) optionally isolating the crystalline ethanolate.

The dissolution in Step A is suitably conducted at a temperature in a range of from about 5° C. to about 30° C., is typically conducted at a temperature in a range of from about 20° C. to about 25° C., and is preferably conducted at a temperature of about 25° C. The concentration of Isomer M in methylene chloride in Step A is suitably in a range of from about 0.01 to about 0.15 g/mL, is typically in a range of from about 0.01 to about 0.1 g/mL, and is preferably about 0.1 g/mL.

The solvent switching in Step B is suitably conducted at a temperature in a range of from about 5° C. to about 25° C., and is preferably conducted at a temperature of from about 5° C. to about 20° C. The concentration of Isomer M in the ethanol slurry in Step B is suitably in a range of from about 0.01 to about 0.2 g/mL, is typically in a range of from about 0.05 to about 0.15 g/mL, and is preferably about 0.1 g/mL.

The slurry is suitably aged in Step C at a temperature in a range of from about 5° C. to about 30° C.; is typically aged at a temperature in a range of from about 5° C. to about 25° C., and is preferably aged at a temperature in a range of from about 20° C. to about 25° C.

The present invention further includes a second process (referred to as Process P2), which is a process for preparing a crystalline hydrate of Isomer M, which comprises:

(A) adding a crystalline ethanolate of Isomer M as defined and described above to water to provide a slurry;

(B) optionally ageing the slurry from Step A to provide the crystalline hydrate; and

(C) optionally isolating the crystalline hydrate.

The addition of crystalline ethanolate in Step A is suitably conducted at a temperature in a range from about 20° C. to about 40° C., is typically conducted at a temperature in a range from about 25° C. to about 35° C., and is preferably conducted at a temperature of about 30° C. The ethanolate is suitably employed in Step A in an amount in a range of from about 1 wt. % to about 20 wt. %, is typically employed in an amount in a range of from about 2 wt. % to about 15 wt. %, and is preferably about 10 wt. %.

The ageing in Step B is suitably conducted at a temperature in a range of from about 5° C. to about 30° C., is typically conducted at a temperature in a range of from about 15° C. to about 25° C., and is preferably conducted at a temperature of about 25° C.

The present invention still further includes a third process (Process P3), which is a process for preparing a crystalline anhydrate of Isomer M, which comprises:

(D) forming a slurry of a crystalline ethanolate of Isomer M as defined and described above in a slurrying agent selected from the group consisting of di-C₁-C₆ alkyl ethers, C₄-C₆ cyclic ethers, C₁-C₆ alkyl acetates and di-C₁-C₆ alkyl ketones, and optionally ageing the slurry, to obtain the crystalline anhydrate; and

(E) optionally isolating the crystalline anhydrate.

The forming and optional ageing of the slurry in Step D are each suitably conducted at a temperature in a range of from about 5° C. to about 30° C., and are each typically conducted at a temperature in a range of from about 15° C. to about 30° C., and are each preferably conducted at a temperature of about 25° C. The slurrying agent is typically selected from the group consisting of MTBE, THF, EtOAc, IPAc, and acetone, and is preferably MTBE. The crystalline ethanolate in Step D is suitably employed in an amount in a range of from about 0.01 g/mL to about 0.2 g/mL of the slurrying agent, and is typically employed in an amount in a range of from about 0.1 g/mL to about 0.15 g/mL.

A first embodiment of Process P3 (alternatively referred to herein as “Embodiment P3-E1”) is Process P3 comprising Steps D and E as set forth above and which further comprises:

(B) reacting the M-atropisomer of 5-10:

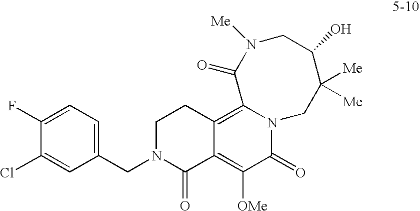

with boron tribromide to obtain Isomer M; and

(C) forming a slurry of Isomer M from Step B in ethanol, and optionally ageing the slurry, to obtain crystalline ethanolate of Isomer M.

The reaction of Step B is suitably conducted in an organic solvent, and is typically conducted in methylene chloride. The reaction of Step B is suitably conducted at a temperature in a range of from about 5° C. to about 30° C., and is typically conducted at a temperature in a range of from about 20° C. to about 25° C. Boron tribromide is suitably employed in Step B in an amount in a range of from about 2.0 to about 4.0 equivalents, and typically in a range of from about 2.1 to about 2.5 equivalents, per equivalent of 5-10.

The forming and optional ageing of the slurry in step C are each suitably conducted at a temperature in a range of from about 5° C. to about 30° C., and are each typically conducted at a temperature in a range of from about 20° C. to about 25° C. Isomer M in Step C is suitably employed in an amount in a range of from about 0.01 g/mL to about 0.2 g/mL of ethanol, and is typically employed in an amount in a range of from about 0.05 g/mL to about 0.15 g/mL.

A second (and preferred) embodiment of the Process P3 (Embodiment P3-E2) is a process comprising Steps B to E as described above, wherein the M-atropisomer 5-10 employed in Step B is crystalline and substantially pure, the crystalline ethanolate of Isomer M resulting from Step C is substantially pure, and the crystalline anhydrate of Isomer M resulting from Step D and optionally isolated in Step E is substantially pure. The term “substantially pure” has the meaning set forth above in Embodiment E18.

A third embodiment of Process P3 (Embodiment P3-E3) is Process P3 as described in Embodiment P3-E1 comprising Steps B, C, D and E, which further comprises:

(A) forming a slurry of a mixture of M- and P-atropisomers of 5-10 in a C₁-C₆ alkyl acetate, optionally ageing the slurry, and separating from the slurry M-atropisomer of 5-10.

The M-atropisomer of 5-10 resulting from Step A is normally crystalline and substantially pure. It was unexpectedly discovered that Step A can provide a substantially pure M-atropisomer of 5-10 wherein essentially all of the P-atropisomer remains in the mother liquor. Accordingly, a sub-embodiment of Embodiment P3-E3 is the process described in Embodiment P3-E3 wherein the M-atropisomer 5-10 resulting from Step A and employed in Step B is crystalline and substantially pure, the crystalline ethanolate of Isomer M resulting from Step C is substantially pure, and the crystalline anhydrate of Isomer M resulting from Step D and optionally isolated in Step E is substantially pure. The term “substantially pure” has the meaning set forth above in Embodiment E18.

The alkyl acetate employed in Step A is typically ethyl acetate or isopropyl acetate, and is preferably ethyl acetate. The forming and optional ageing of the slurry in Step A are each suitably conducted at a temperature in a range of from about 5° C. to about 30° C., and are each typically conducted at a temperature in a range of from about 20° C. to about 25° C. The amount of 5-10 employed in the slurry in Step A is suitably in a range of from about 5 g/mL to about 15 g/mL of alkyl acetate.

The term “aging” and variants thereof (e.g., “aged”) as used in Processes P1, P2 and P3 mean maintaining the slurry for a time and under conditions effective to provide a higher yield of the desired crystalline form compared to that which can be achieved in the absence of ageing. Effective conditions include conducting the ageing in a suitable temperature range and optionally but preferably with a suitable degree of agitation (e.g., stirring). The ageing step is optional in the sense that at least some of the desired material forms during the slurrying step (e.g., during the solvent switch in Step B of Process P1 or during the formation of the slurry in Step A of Process P2), but inclusion of an ageing step is preferred in order to improve, and preferably maximize, yield.

The optional isolation step in each of the foregoing processes means recovery of the resulting crystalline product from the slurry. Isolation of the crystalline product can be accomplished, for example, by separation of the crystalline material by filtration, washing the filtered crystalline product with the slurrying agent (e.g., with ethanol in Process P1 and with water in Process P2), and then drying the washed product with low heat (e.g., at a temperature in a range of from about 30° C. to about 40° C.) and/or low vacuum.

Unless expressly stated to the contrary, all ranges set forth herein are inclusive. Thus, for example, when a temperature is said to be in a range of from about 5° C. to about 30° C., it means the temperature can be about 5° C. or about 30° C. or any temperature in between.

Abbreviations used herein include the following: A %=area percent in an HPLC scan; AIDS=acquired immunodeficiency syndrome; ARC=AIDS related complex; AUC=area under the curve of plasma concentration versus time; Cₘₐₓ=peak plasma concentration; CPMAS=cross-polarization magic angle spinning NMR; DABCO=1,4-diazabicyclo[2.2.2]octene; DMAc=N,N-dimethylacetamide; DMF=N,N-dimethylformamide; DSC=differential scanning calorimetry; EDC=1-ethyl-3-(3-dimethylaminopropyl) carbodiimide; ES MS=electrospray mass spectroscopy; EtOAc=ethyl acetate; FBS=fetal bovine serum; g=gram(s); HIV=human immunodeficiency virus; HOAt=1-hydroxy-7-azabenzotriazole; HPLC=high performance liquid chromatography; IPAc=isopropyl acetate; KF=Karl Fisher titration for water; LC=liquid chromatography; Me=methyl; MeOH=methanol; Ms=mesyl or methanesulfonyl; MTBE=methyl t-butyl ether; NMR=nuclear magnetic resonance; Red-Al=Sodium bis(2-methoxyethoxy)aluminum hydride; SE=standard error; SPINAL=small phase incremental alternation; t-Bu=tertiary butyl; TFA=trifluoroacetic acid; TGA=thermogravimetric analysis; THF=tetrahydrofuran; OTHP=tetrahydropyran-2-yloxy; XRPD=x-ray powder diffraction.

Unless expressly stated to the contrary or otherwise clear from the context, references to equivalents (eqs.) mean molar equivalents.

The following examples serve only to illustrate the invention and its practice. The examples are not to be construed as limitations on the scope or spirit of the invention.

### EXAMPLE 1

### Isomers M and P of (4R)-11-(3-Chloro-4-fluorobenzyl)-4,9-dihydroxy-2,5,5-trimethyl-3,4,5,6,12,13-hexahydro-2H[1,4]diazocino[2,1-a]-2,6-naphthyridine-1,8,10(11H)-trione

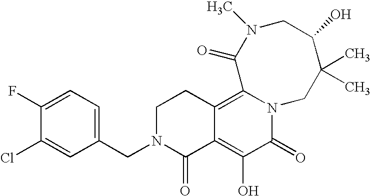

Step 1: 1-(3-Chloro-4-fluorobenzyl)piperidin-2-one

To a cold (0° C.) solution of valerolactam (153.30 g, 1.54 mol) in mixture of anhydrous 1-methyl-2-pyrrolidinone (3.5 L) and THF (350 mL), sodium hydride (67.7 g, 1.69 mol, 60% dispersion in oil) was added over a period of 5 minutes. The reaction mixture was stirred for 30 minutes, and a solution of 3-chloro-4-fluorobenzylbromide (345.5 g, 1.54 mol) in 1-methyl-2-pyrrolidinone (200 mL) was added over 30 minutes at 0° C. The reaction mixture was stirred at 0° C. for 1 hour, and was allowed to warm up and stirred at room temperature overnight. The reaction mixture was quenched with distilled water (5 L), and extracted with dichloromethane (three times; 2 L, 1 L, 1 L). The organic extracts were combined, washed with water (3×; 4 L each time). The residual oil was dissolved in ethyl acetate (4 L), and extracted with water (3×; 2 L each time). The organic layer was separated, concentrated under vacuum to give the title product that solidified upon standing.

¹H NMR (400 MHz, CDCl₃) δ 7.24 (m, 2H), 7.0 (m, 2H), 7.1 (m, 1H), 4.56 (s, 2H), 3.19 (t, J=4.9 Hz, 2H), 2.46 (t, J=6.4 Hz, 2H), 1.8-1.75 (m, 4H).

Step 2: 1-(3-Chloro-4-fluorobenzyl)-5,6-dihydropyridin-2(1H)-one

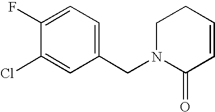

To a cold (−20° C.) solution of 1-(3-chloro-4-fluorobenzyl)piperidin-2-one (340 g, 1.41 mol) in anhydrous tetrahydrofuran (5 L) under an atmosphere of nitrogen, a solution of lithium bis(trimethylsilyl)amide (3.09 L, 3.09 mol; 1M in THF) was added over a period of 40 minutes with the temperature of the reaction maintained at −20° C. After the addition was complete, the reaction mixture was stirred at −20° C. for one hour. Methyl benzene sulfonate (231 mL, 1.69 mol) was added to the reaction mixture over a period of 30 minutes. The reaction mixture was stirred at −20° C. for 30 minutes. The product mixture was diluted with ethyl acetate (4 L) and washed with water (four times; 2 L each time). The organic extract was concentrated under vacuum. The residue was dissolved in toluene (4 L), treated with solid sodium carbonate (500 g), and heated at 100° C. for one hour. The product mixture was diluted with ethyl acetate (4 L) and washed with water (4 times; 2 L each). The organic extract was concentrated under vacuum. The residue was subjected to column chromatography on silica gel eluting with a gradient of 0-60% EtOAc in heptane. Collection and concentration of appropriate fractions provide the title compound as oil.

¹H NMR (400 MHz, CDCl₃) δ 7.3 (m, 1H), 7.15 (m, 1H), 7.1 (t, 1H), 6.6 (m, 1H), 6.0 (m, 1H), 4.55 (s, 2H), 3.33 (t, 2H), 1.38 (m, 2H). ES MS M+1=240.13

Step 3: 2-Butoxy-2-oxoethanaminium chloride

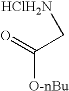

To a suspension of glycine hydrochloride (400 g, 3.58 mol) in n-butanol (8 L), thionyl chloride (1.37 L, 18.84 mol) was added slowly dropwise. After addition was complete, the reaction was heated at 70° C. overnight. The product mixture was concentrated under vacuum and the residue was triturated with a mixture of heptane/ethyl acetate. The white solid precipitated was filtered and dried under a stream of dry nitrogen to provide the title compound.

¹H NMR (400 MHz, CDCl₃) δ 8.5 (br s, 3H), 4.18 (t, J=6.7 Hz, 2H), 4.0 (br s, 2H), 1.62 (m, 2H), 1.38 (m, 2H), 0.92 (t, J=7.4 Hz, 3H). ES MS M+1=132.

Step 4: Butyl N-[ethoxy(oxo)acetyl]glycinate

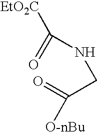

A mixture of 2-butoxy-2-oxoethanaminium chloride (573.5 g, 3.42 mol), triethylamine (415 g, 4.1 mol), and diethyl oxalate (1.0 kg, 6.8 mol) in ethanol (7 L) was heated at 50° C. for 3 hours. The product mixture was cooled and concentrated under vacuum. The residue was dissolved in methylene chloride and washed with two 4 L portions of water. The organic fraction was dried over anhydrous magnesium sulfate, filtered, and concentrated under vacuum. The residual oil was subjected to column chromatography on silica gel eluting with heptane/ethyl acetate gradient. Collection and concentration of appropriate fractions provided the title material.

¹H NMR (400 MHz, CDCl₃) δ 7.56 (br s, 1H), 4.37 (q, J=7.2 Hz, 2H), 4.2 (t, J=6.6 Hz, 2H), 4.12 (d, J=5.5 Hz, 2H), 1.64 (p, J=6.8 Hz, 2H), 1.39 (t, J=7.15 Hz, 3H), 1.37 (m, 2H), 0.94 (t, J=7.4 Hz, 3H). ES MS M+1=232.

Alternative route. The glycinate was also prepared using ethyl oxalyl chloride in place of diethyl oxalate as follows: To a mixture of 2-butoxy-2-oxoethanaminium chloride (1.48 Kg, 8.85 mol), dichloromethane (10.6 L), and deionized water (10.6 L) at room temperature, potassium bicarbonate (2.2 Kg, 22.1 mol) was added in three portions. The endothermic mixture was warmed back to 16° C. Ethyl oxalyl chloride (1.08 L, 9.74 mol) was added via an addition funnel over 45 minutes, and stirred at room temperature for two hours. The aqueous layer was separated and extracted with dichloromethane (2×2 L). The organic fractions were combined, and washed with a mixture of deionized water (10 L) and brine (1.5 L). The organic fraction was concentrated under vacuum to provide the title material.

¹H NMR (400 MHz, CDCl₃) δ 7.56 (br s, 1H), 4.37 (q, J=7.2 Hz, 2H), 4.2 (t, J=6.6 Hz, 2H), 4.12 (d, J=5.5 Hz, 2H), 1.64 (p, J=6.8 Hz, 2H), 1.39 (t, J=7.15 Hz, 3H), 1.37 (m, 2H), 0.94 (t, J=7.4 Hz, 3H). ES MS M+1=232.

Step 5: Ethyl 5-butoxy-1,3-oxazole-2-carboxylate

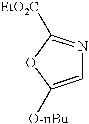

To a solution of butyl N-[ethoxy(oxo)acetyl]glycinate (783 g, 3.38 mol) in acetonitrile (8 L) in a 50 L glass reactor with overhead stirrer, phosphorus pentoxide (415 g, 2.92 mol) was added in portions. The reaction was heated at 60° C. for 1 hour. The product mixture was cooled, and water (8 L) was added with the mixture maintained at 20° C. The resultant mixture was extracted with dichloromethane (8 L, and 3 times 2 L). The organic extracts were combined, washed twice with saturated aqueous sodium bicarbonate (8 L total), dried over anhydrous magnesium sulfate, filtered and concentrated under vacuum. The residual oil was subjected to column chromatography on silica gel eluting with 0-30% heptane/ethyl acetate gradient. Collection and concentration of appropriate fractions provided the title material.

¹H NMR (400 MHz, CDCl₃) δ 6.33 (s, 1H), 4.42 (q, J=7.2 Hz, 2H), 4.18 (t, J=6.4 Hz, 2H), 1.8 (p, J=6.4 Hz, 2H), 1.47 (p, J=7.4 Hz, 2H), 1.41 (t, J=7.15 Hz, 3H), 0.97 (t, J=7.4 Hz, 3H). ES MS M+1=214.

Step 6: Ethyl 6-(3-chloro-4-fluorobenzyl)-4-hydroxy-5-oxo-5,6,7,8-tetrahydro-2,6-naphthyridine-1-carboxylate

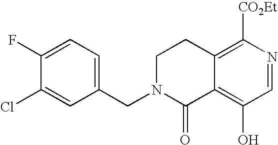

A mixture of ethyl 5-butoxy-1,3-oxazole-2-carboxylate (248 g, 1.16 mol; step 5), 1-(3-chloro-4-fluorobenzyl)-5,6-dihydropyridin-2(1H)-one (199.2 g, 0.83 mol; step 2), and deionized water (22.5 mL, 1.25 mol) in a glass liner of a stainless steel high pressure reactor (with the interstitial space between the liner and the pressure vessel was filled with water) was heated at 135° C. with stirring for 72 hours. The product mixture was cooled in an ice-water bath and the gaseous by-product was carefully vented. The orange solid product was triturated with methyl tert-butyl ether (300 mL) and collected by filtration. The product recrystallized from boiling ethanol-water (˜500 mL, 9:1 v/v), collected by filtration, washed successively with a small quantity of ethanol, methyl tert-butyl ether (300 mL), and heptane (200 mL), and air dried to afford the title compound.

¹H NMR (400 MHz, CDCl₃) δ 12.79 (s, 1H), 8.42 (s, 1H), 7.4 (dd, J=2, 7 Hz, 1H), 7.2 (m, 1H), 7.15 (t, J=8.6 Hz, 1H), 4.7 (s, 2H), 4.4 (q, J=7 Hz, 2H), 3.5 (m, 4H), 1.4 (t, J=7 Hz, 3H). (ES MS M+1=379.0)

Step 7: Ethyl 6-(3-chloro-4-fluorobenzyl)-4-methoxy-5-oxo-5,6,7,8-tetrahydro-2,6-naphthyridine-1-carboxylate

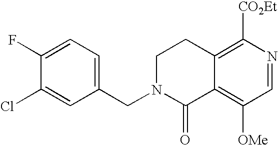

To a stirred solution of ethyl 6-(3-chloro-4-fluorobenzyl)-4-hydroxy-5-oxo-5,6,7,8-tetrahydro-2,6-naphthyridine-1-carboxylate (208 g, 0.55 mol) in a mixture of dichloromethane (830 mL) and methanol (410 mL) at 10° C., a solution of (trimethyl-silyl)diazomethane (600 mL, 1.2 mol; 2M) in hexanes was added over a period of 1 hour with the reaction temperature maintained below 15° C. The reaction mixture (unstirred) was allowed to stand at 10° C. overnight, and then at 20° C. for additional 4 hours. The reaction mixture was cooled back to 10° C. and quenched with acetic acid (˜75 mL). The product mixture was concentrated under vacuum and the residue recrystallized from boiling methyl tert-butyl ether and heptane. The solid recrystallized was collected by filtration, washed with a mixture of methyl tert-butyl ether and heptane (1:1, v/v), and air dried to afford the title compound.

¹H NMR (400 MHz, CDCl₃) δ 8.42 (s, 1H), 7.41 (dd, J=2, 7 Hz, 1H), 7.24 (m, 1H), 7.11 (t, J=8.6 Hz, 1H), 4.70 (s, 2H), 4.42 (q, J=7 Hz, 2H), 4.12 (s, 3H), 3.4 (m, 4H), 1.42 (t, J=7 Hz, 3H). (ES MS M+1=392.9)

Step 8: Ethyl 3-(acetyloxy)-6-(3-chloro-4-fluorobenzyl)-4-methoxy-5-oxo-5,6,7,8-tetrahydro-2,6-naphthyridine-1-carboxylate

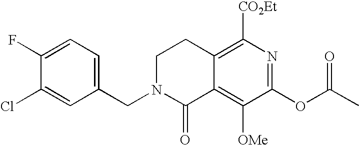

To a cold (5° C.) mixture of ethyl 6-(3-chloro-4-fluorobenzyl)-4-methoxy-5-oxo-5,6,7,8-tetrahydro-2,6-naphthyridine-1-carboxylate (199 g, 0.51 mol) and urea hydrogen peroxide (100 g, 1.06 mol) in dichloromethane (1.5 L), trifluoroacetic anhydride was added dropwise over a period of 45 minutes. The resultant homogeneous solution was stirred at 20° C. for 30 minutes and cooled back to 5° C. The reaction mixture was treated with aqueous potassium hydrogen phosphate (pH of aqueous extract increased to ˜8), followed by slow addition of freshly prepared aqueous sodium bisulfite solution with the temperature of the product mixture maintained below 25° C. The organic extract was separated and the aqueous fraction extracted with toluene (2×). The organic extracts were combined, dried over anhydrous sodium sulfate, filtered, and concentrated under vacuum. Without further purification, a solution of this intermediate N-oxide (˜280 g) and acetic anhydride (239 mL, 2.5 mol) in toluene (2 L) was heated at 110° C. for 16 hours. The product mixture was concentrated under vacuum. The resultant oil was concentrated from toluene (300 mL, twice) and stored under vacuum overnight. The acetate product was used in the following step without further purification.

(ES MS M+1=408.9)

Step 9: 6-(3-Chloro-4-fluorobenzyl)-4-methoxy-3,5-dioxo-2,3,5,6,7,8-hexahydro-2,6-naphthyridine-1-carboxylic acid

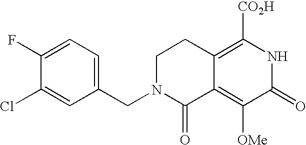

A mixture of ethyl 3-(acetyloxy)-6-(3-chloro-4-fluorobenzyl)-4-methoxy-5-oxo-5,6,7,8-tetrahydro-2,6-naphthyridine-1-carboxylate (217 g, 0.48 mol), lithium hydroxide monohydrate (70.7 g, 1.67 mol), and water (320 mL) in ethanol (1.8 L) was sonicated for 20 minutes. The reaction mixture was cooled in an ice-water bath and treated with hydrochloric acid (425 mL, 3 M). The resultant light yellow solid was filtered, washed successively with water (1 L), a 3:2 v/v mixture of water and ethanol (500 mL), MTBE (750 mL), and air dried. The yellow solid was dissolved in anhydrous DMF (700 mL) and concentrated under vacuum. The procedure was repeated twice to remove residual water. The yellow solid was triturated with MTBE, filtered, and stored under vacuum overnight to afford the title acid.

¹H NMR (400 MHz, CDCl₃) δ 7.54 (dd, J=2, 7 Hz, 1H), 7.3 (m, 2H), 4.65 (s, 2H), 3.89 (s, 3H), 3.43 (t, J=5.5 Hz, 2H), 3.00 (t, J=5.5 Hz, 2H). (ES MS M+1=380.9)

Step 10: (3R)-4,4-Dimethyl-3-(tetrahydro-2H-pyran-2-yloxy)dihydrofuran-2(3H)-one

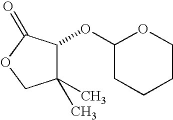

To a mixture of D(−)-pantolactone (10.0 g, 76.8 mmol) and p-toluenesulfonic acid monohydrate (0.1 g, 0.5 mmol) in anhydrous methylene chloride (130 mL) under an atmosphere of nitrogen at room temperature, 3,4-dihydro-2H-pyran was added dropwise over a period of 20 minutes. (See Ito et al., Synthesis 1993, pp 137-140; Szabo et al., Tetrahedron Asymmetry 1999, 10: pp 61-76). The reaction mixture was stirred at the same temperature for 45 minutes. The product mixture was treated with water (150 mL) and diluted with dichloromethane (150 mL). The organic extract was washed with brine, dried over anhydrous magnesium sulfate, filtered, and concentrated under vacuum. The residue was subjected to purification on silica gel eluting with 0-40% ethyl acetate in hexane gradient. Collection and concentration of appropriate fractions provided the title compound as a mixture of diastereomers.

¹H NMR (400 MHz, CDCl₃) δ 5.16 (t, J=3.7 Hz, 0.73H), 4.86 (t, J=2.9 Hz, 0.27H), 5.24-3.53 (m), 1.22 (s, 2.2H), 1.20 (s, 0.8H), 1.14 (s, 2.2H), 1.11 (s, 0.8H).

Step 11: (2R)-4-Hydroxy-N,3,3-trimethyl-2-(tetrahydro-2H-pyran-2-yloxy)-butanamide

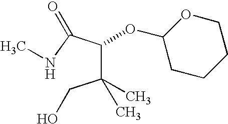

To a cold (0° C.) solution of methylamine in methanol (7.6 mL; 40% aqueous solution) in methanol (70 mL), (3R)-4,4-Dimethyl-3-(tetrahydro-2H-pyran-2-yloxy)dihydrofuran-2(3H)-one (15 g, 70 mmol) was added. The reaction mixture was stirred at the room temperature for 3 hours. The product mixture was concentrated under vacuum. The residue was subjected to purification on silica gel eluting with 20-100% ethyl acetate in hexane gradient. Collection and concentration of appropriate fractions provided the title compound as a mixture of diastereomers.

¹H NMR (400 MHz, CDCl₃) δ 6.76 (br signal, 0.27H), 6.35 (br signal, 0.73H), 4.38-3.18 (m), 2.86 (d, J=5.1 Hz, 2.2H), 2.85 (d, J=5.6 Hz, 0.8H), 1.03 (s, 3H), 0.88 (s, 3H).

Step 12: (3R)-2,3-Dimethyl-4-(methylamino)-3-(tetrahydro-2H-pyran-2-yloxy)-butan-1-ol

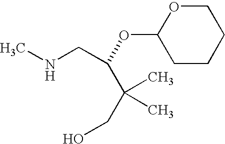

A solution of (2R)-4-Hydroxy-N,3,3-trimethyl-2-(tetrahydro-2H-pyran-2-yloxy)-butanamide (11.6 g, 47.3 mmol) in anhydrous THF (90 mL) under an atmosphere of nitrogen was treated with a solution of lithium aluminum hydride in THF (142 mL, 1M, 142 mmol). The reaction mixture was heated in an oil bath at 77° C. for 72 hours. The product mixture was cooled with an ice-water bath and was treated successively with water (5.4 mL), 15% aqueous NaOH (5.4 mL), and water (16.2 mL). The resultant slurry was stirred at room temperature for 1 hour and filtered through a pad of Celite. The solid was washed with THF. The combined filtrate was dried over anhydrous sodium sulfate, filtered, and concentrated under vacuum. The residue was concentrated from benzene under vacuum to afford the title compound as a mixture of diastereomers.

¹H NMR (400 MHz, CDCl₃) δ 4.68 (m, 0.73H), 4.47 (m, 0.27H), 3.9-2.4 (m), 2.46 (s, 0.8H), 2.43 (s, 2.2H), 0.97 (s, 0.8H), 0.95 (s, 2.2H), 0.93 (s, 2.2H), 0.85 (s, 0.8H).

ES-MS M+1=232.

Step 13: 6-(3-Chloro-4-fluorobenzyl)-N-[(2R)-4-hydroxy-3,3-dimethyl-2-(tetrahydro-2H-pyran-2-yloxy)butyl]-4-methoxy-N-methyl-3,5-dioxo-2,3,5,6,7,8-hexahydro-2,6-naphthyridine-1-carboxamide

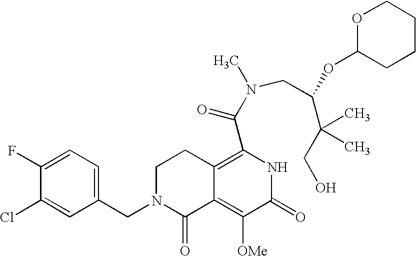

A mixture of 6-(3-chloro-4-fluorobenzyl)-4-methoxy-3,5-dioxo-2,3,5,6,7,8-hexahydro-2,6-naphthyridine-1-carboxylic acid (15.8 g, 41.5 mmol), (3R)-2,3-dimethyl-4-(methylamino)-3-(tetrahydro-2H-pyran-2-yloxy)-butan-1-ol (9.6 g, 41.5 mmol), EDC (9.6 g, 49.8 mmol), HOAt (0.28 g, 2.1 mmol) and N-methylmorpholine (22.9 mL, 207 mmol) in anhydrous methylene chloride (300 mL) was stirred at room temperature overnight. The product solution was diluted with methylene chloride and washed successively with water and brine. The organic extract was dried over anhydrous sodium sulfate, filtered, and concentrated under vacuum. The residue was subjected to column chromatography on silica gel eluting with 0-10% methanol/chloroform gradient. Collection and concentration of appropriate fractions provided the title material as a mixture of two diastereomers. ES-MS M+H=594 for both isomers.

Step 14: (4R)-11-(3-Chloro-4-fluorobenzyl)-9-methoxy-2,5,5-trimethyl-4-(tetrahydro-2H-pyran-2-yloxy)-3,4,5,6,12,13-hexahydro-2H[1,4]diazocino[2,1-a]-2,6-naphthyridine-1,8,10(11H)-trione

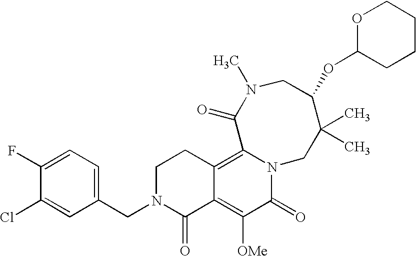

To a solution of 6-(3-chloro-4-fluorobenzyl)-N-[(2R)-4-hydroxy-3,3-dimethyl-2-(tetrahydro-2H-pyran-2-yloxy)butyl]-4-methoxy-N-methyl-3,5-dioxo-2,3,5,6,7,8-hexahydro-2,6-naphthyridine-1-carboxamide (4.0 g, 6.7 mmol) and diisopropylethylamine (2.6 mL, 14.8 mmol) in dichloromethane (34 mL) at room temperature, methanesulfonic anhydride (2.3 g, 13.5 mmol) was added. The reaction mixture was stirred at room temperature for 1 hour. The product mixture was diluted with methylene chloride and washed with water. The organic extract was dried over anhydrous sodium sulfate, filtered, and concentrated under vacuum. This intermediate mixture of mono- and bis-mesylates was used in the following cyclization reaction without further purification.

A mixture of the above mesylates (0.88 g, 1.17 mmol) and cesium carbonate (1.52 g, 4.69 mmol) in anhydrous DMF (18 mL) was heated in a microwave oven at 150° C. for 30 minutes. The reaction mixture was filtered through a pad of Celite and the solid filtered washed with DMF. Filtrates from five consecutive runs were combined and concentrated under vacuum. The residue was partitioned between ethyl acetate and brine. The organic extract was washed with brine, dried over anhydrous sodium sulfate, filtered and concentrated under vacuum. The residue was subjected to column chromatography on silica gel eluting with 0-5% methanol/chloroform gradient. Collection and concentration of appropriate fractions afforded the title compound as a mixture of two diastereomers.

¹H NMR (400 MHz, CDCl₃) δ 7.37 (dd, J=1.8, 6.8 Hz, 1H), 7.21 (m, 1H), 7.10 (t, J=8.6 Hz, 1H), 4.86-4.49 (m),4.08 (s), 4.07 (s), 4.2-2.8 (m), 3.17 (s), 3.13 (s), 1.8-1.4 (br m), 1.25 (s), 1.10 (s), 0.95 (s), 0.91 (s). ES-MS M+H=576 for both isomers.

Alternative route. The title intermediate was also prepared as follows: To a solution of 6-(3-chloro-4-fluorobenzyl)-N-[(2R)-4-hydroxy-3,3-dimethyl-2-(tetrahydro-2H-pyran-2-yloxy)butyl]-4-methoxy-N-methyl-3,5-dioxo-2,3,5,6,7,8-hexahydro-2,6-naphthyridine-1-carboxamide (20.0 g, 33.7 mmol) and diisopropylethylamine (12.9 mL, 74.1 mmol) in dichloromethane (168 mL) at room temperature, methanesulfonic anhydride (12.3 g, 70.7 mmol) was added dropwise. The exothermic reaction was cooled with an ice-water bath. The reaction mixture was stirred at room temperature for 1 hour. The product mixture was diluted with methylene chloride and washed with water. The organic extract was dried over anhydrous sodium sulfate, filtered, and concentrated under vacuum. This intermediate mixture of mono- and bis-mesylates was used in the following cyclization reaction without further purification.

A mixture of the above mesylates (13.1 g) and cesium carbonate (13.1 g, 40 mmol) in anhydrous DMF (600 mL) was heated at 105° C. for 6 hours with vigorous stirring. The reaction mixture was filtered through a pad of Celite and the solid filtered washed with DMF. The filtrates were combined and concentrated under vacuum. The residue was partitioned between ethyl acetate and brine. The organic extract was washed with brine, dried over anhydrous sodium sulfate, filtered and concentrated under vacuum to provide the title compound as a mixture of two diastereomers. The mixture was used in the following step without any further purification.

¹H NMR (400 MHz, CDCl₃) δ 7.37 (dd, J=1.8, 6.8 Hz, 1H), 7.21 (m, 1H), 7.10 (t, J=8.6 Hz, 1H), 4.86-4.49 (m),4.08 (s), 4.07 (s), 4.2-2.8 (m), 3.17 (s), 3.13 (s), 1.8-1.4 (br m), 1.25 (s), 1.10 (s), 0.95 (s), 0.91 (s). ES-MS M+H=576 for both isomers.

Step 15: (4R)-11-(3-Chloro-4-fluorobenzyl)-4,9-dihydroxy-2,5,5-trimethyl-3,4,5,6,12,13-hexahydro-2H[1,4]diazocino[2,1-a]-2,6-naphthyridine-1,8,10(11H)-trione

To a cold (0° C.) solution of (4R)-11-(3-chloro-4-fluorobenzyl)-9-methoxy-2,5,5-trimethyl-4-(tetrahydro-2H-pyran-2-yloxy)-3,4,5,6,12,13-hexahydro-2H[1,4]diazocino[2,1-a]-2,6-naphthyridine-1,8,10(11H)-trione (3.3 g, 5.7 mmol) in anhydrous methylene chloride (35 mL), solution of boron tribromide in methylene chloride (22.9 mL, 1.0 M, 22.9 mmol) was added. The reaction mixture was stirred at room temperature for 2 hours. Reaction mixture was cooled with an ice-water bath, quenched with water (20 mL), and stirred at room temperature for 30 minutes. The product mixture was diluted with methylene chloride (100 mL) and water (50 mL). Small amount of methanol was added to dissolve the gummy material in the organic phase. The aqueous phase was separated and extracted with methylene chloride. The organic extracts were combined and washed with brine, dried over anhydrous magnesium sulfate, filtered, and concentrated under vacuum. The residue was purified with preparative HPLC on a 50×250 mm Xterra 10 micron column eluted with a 20-35% acetonitrile-water gradient at 100 mL/minute over 50 minutes. Fractions of the faster eluting major isomer were collected and lyophilized to afford the major isomer as an amorphous white solid.

Isomer M: ¹H NMR (500 MHz, CDCl₃) δ 13.1 (br s, 1H), 7.35 (dd, J=2.2, 6.8 Hz, 1H), 7.20 (m, 1H), 7.13 (t, J=8.8 Hz, 1H), 4.84 (d, J=14.6 Hz, 1H), 4.74 (d, J=14.6 Hz, 1H), 4.59 (d, J=14.6 Hz, 1H), 3.73 (dd, J=14.9, 9.5 Hz, 1H), 3.43 (m, 3H), 3.18 (s, 3H), 3.13 (d, J=14.6 Hz, 1H), 3.01 (d, J=14.9 Hz, 1H), 2.92 (m, 1H), 2.52 (dt, J=15.6, 4.9 Hz, 1H), 1.21 (s, 3H), 0.94 (s, 3H). (ES MS M+1=478.1).

Fractions of the slower eluting minor isomer were collected and lyophilized. The solid was further purified with preparative HPLC on a 50×250 mm Xterra 10 micron column eluted with a 20-37% acetonitrile-water gradient at 100 mL/minute over 50 minutes. Collection and lyophilization of appropriate fractions provided the minor isomer as pale yellow solid.

Isomer P: ¹H NMR (500 MHz, CDCl₃) δ 13.0 (br s, 1H), 7.36 (dd, J=2.0, 6.8 Hz, 1H), 7.20 (m, 1H), 7.13 (t, J=8.6 Hz, 1H), 4.76 (d, J=14.6 Hz, 1H), 4.60 (d, J=10.0 Hz, 1H), 4.57 (d, J=10.0 Hz, 1H), 3.74 (d, J=14.9 Hz, 1H), 3.61 (d, J=14.4 Hz, 1H), 3.45-3.35 (m, 4H), 3.25 (s, 3H), 2.92 (m, 1H), 2.48 (dt, J=15.8, 4.6 Hz, 1H), 1.15 (s, 3H), 0.87 (s, 3H). (ES MS M+1=478.2).

From NMR studies conducted with pure samples of the individual isomers M and P dissolved in CD2Cl₂ for over 24 hours, it was determined that Isomers M and P are related as diastereomers due to the presence of the chiral (R) hydroxy group in the 8-membered ring and the different orientations (i.e., atropisomerism) of the amide group in the 8-membered ring as a result of the restricted rotation of this amide group relative to the bicyclic core. No equilibration between the two isomers was observed in the NMR studies. Adopting the Helical nomenclature for assigning atropisomers (see Prelog et al., Angew. Chem. Int. Ed. Engl. 1992, 21: 567-583) Isomer M is M-(4R)-11-(3-chloro-4-fluorobenzyl)-4,9-dihydroxy-2,5,5-trimethyl-3,4,5,6,12,13-hexahydro-2H[1,4]diazocino[2,1-a]-2,6-naphthyridine-1,8,10(11H)-trione; and Isomer P is P-(4R)-11-(3-chloro-4-fluorobenzyl)-4,9-dihydroxy-2,5,5-trimethyl-3,4,5,6,12,13-hexahydro-2H[1,4]diazocino[2,1-a]-2,6-naphthyridine-1,8,10(11H)-trione.

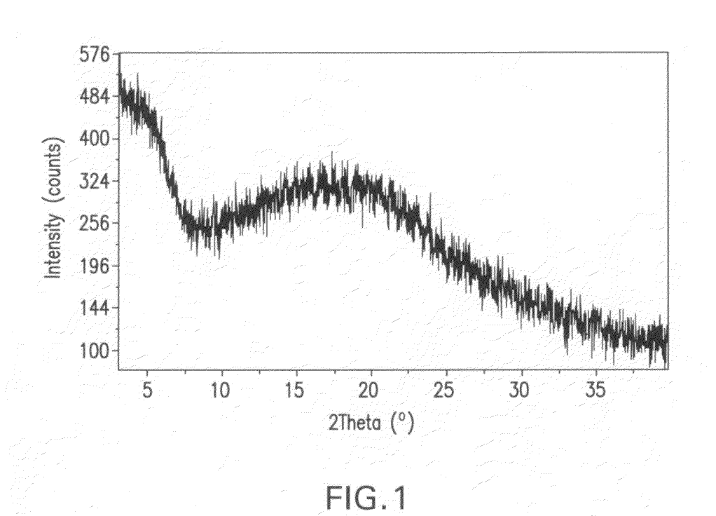

An XRPD pattern of Isomer M prepared in accordance with the method described above was generated using the same instrument and procedures as described in Part B of Example 2. The pattern is shown in FIG. 1. The pattern shows a very broad, featureless band having an absence of peaks due to the amorphous nature of the material.

### EXAMPLE 2

### Crystalline ethanolate of Isomer M

### Part A: Preparation

Isomer M (10 g) was dissolved in 100 mL of CH₂Cl₂ and then the solution was concentrated under reduced pressure (40-60 mm Hg) with feeding of EtOH to keep a constant total volume of 100 mL until all of the CH₂Cl₂ was removed. Crystals began to form during the solvent switch and the slurry resulting from the switch was aged at 25° C. with stirring for two hours. The crystalline material was then separated from the slurry by filtration, washed with ethanol (50 mL), and dried in an oven at 40° C. for 12 hours to provide the title product, which was determined to be a crystalline ethanolate by the characterization studies described below.

### Part B: Characterization

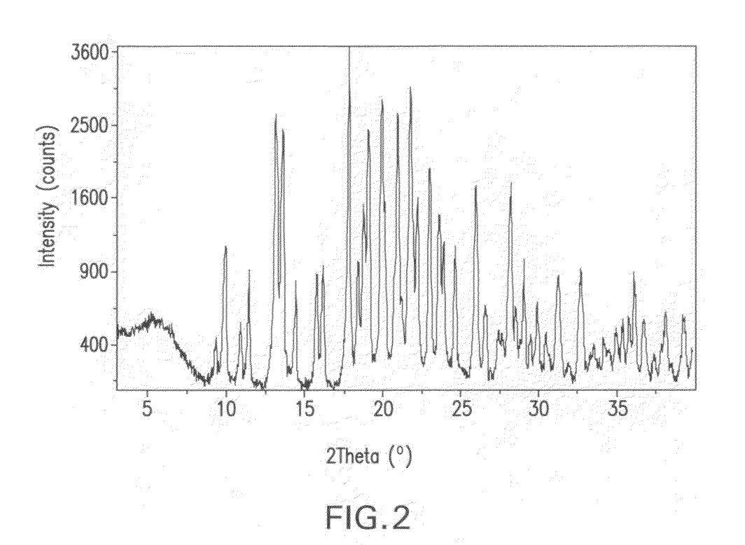

An XRPD pattern of crystalline ethanolate prepared in accordance with the method described in Part A was generated on a Philips Pananalytical X'Pert Pro X-ray powder diffractometer with a PW3040/60 console using a continuous scan from 2.5 to 40 degrees 2Θ. Copper K-Alpha 1 (K_{α1}) and K-Alpha 2 (K_{α2}) radiation was used as the source. The experiment was conducted with the sample at room temperature and open to the atmosphere. The XRPD pattern is shown in FIG. 2. 2Θ values, the corresponding d-spacings, and the relative peak intensities in the XRPD pattern include the following:

TABLE 2
XRPD of crystalline ethanolate

| Peak No. | d-spacing (Å) | 2 Theta | I/Imax (%) |
|---|---|---|---|
| 1 | 3.1 | 29.1 | 23 |
| 2 | 3.2 | 28.2 | 45 |
| 3 | 3.4 | 26.0 | 44 |
| 4 | 3.6 | 24.7 | 24 |
| 5 | 3.7 | 23.9 | 26 |
| 6 | 3.8 | 23.6 | 35 |
| 7 | 3.9 | 23.0 | 50 |
| 8 | 4.0 | 22.2 | 40 |
| 9 | 4.1 | 21.8 | 81 |
| 10 | 4.2 | 21.0 | 70 |
| 11 | 4.4 | 20.1 | 38 |
| 12 | 4.5 | 19.9 | 77 |
| 13 | 4.7 | 19.1 | 64 |
| 14 | 4.8 | 18.4 | 22 |
| 15 | 5.0 | 17.8 | 100 |
| 16 | 5.5 | 16.2 | 22 |
| 17 | 6.5 | 13.6 | 65 |
| 18 | 6.7 | 13.2 | 70 |
| 19 | 8.9 | 9.9 | 26 |

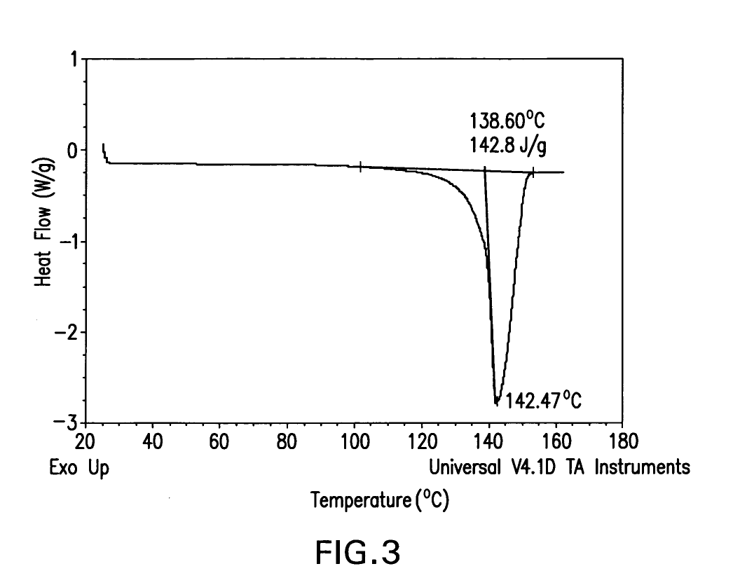

Crystalline ethanolate prepared in accordance with the method described in Part A was also analyzed with a TA Instruments DSC Q 1000 differential scanning calorimeter (DSC) at a heating rate of 10° C./minute from 25° C. to 160° C. in an open aluminum pan in a nitrogen atmosphere. The DSC curve (see FIG. 3) exhibited an endotherm with an onset temperature of 139° C. and a peak temperature of 142° C. The enthalpy change was 142 J/g. The endotherm is believed to be due to melting.

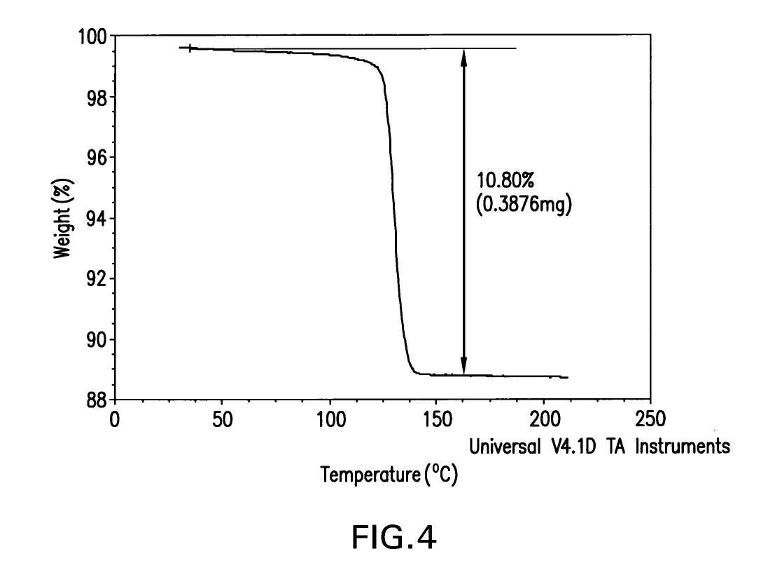

A thermogravimetric analysis (TGA) of crystalline ethanolate prepared in accordance with the method described in Part A was performed with a TA Instruments TGA Q 500 under nitrogen at a heating rate of 10° C./minute from 25° C. to 225° C. The TG curve (see FIG. 4) showed a weight loss of 10.8 wt. % up to 160° C.

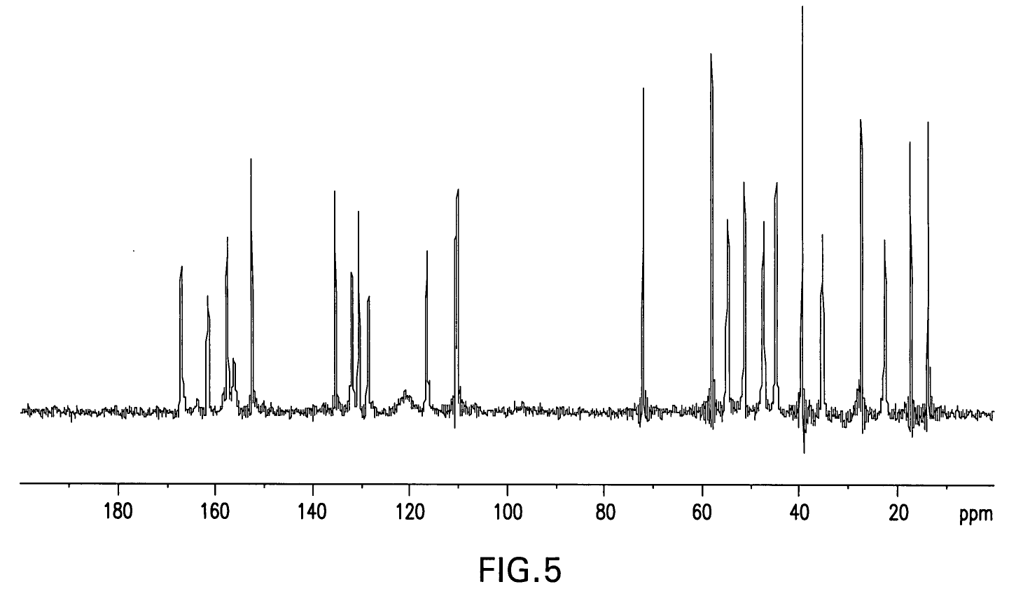

The crystalline ethanolate was also characterized by solid-state carbon-13 NMR spectroscopy. The spectrum was obtained on a Bruker DSX 500 WB NMR system using a Bruker 4 mm H/XIY CPMAS probe. The carbon-13 NMR spectrum utilized proton/carbon-13 cross polarization magic-angle spinning with variable amplitude cross polarization, total sideband suppression, and SPINAL decoupling at 100 kHz. The sample was spun at 10.0 kHz, and a total of 256 scans were collected with a recycle delay of 3 seconds. A line broadening of 10 Hz was applied to the spectrum before FT was performed. Chemical shifts are reported on the TMS scale using the carbonyl carbon of glycine (176.03 ppm) as a secondary reference. The spectrum is shown in FIG. 5, and chemical shift values are presented in the following table:

TABLE 3
Chemical shifts in the solid-state carbon-13 CPMAS NMR
spectrum of the crystalline ethanolate

| | | Chemical Shift |
|---|---|---|
| Peak No. | I/Imax (%) | (ppm) |
| 1 | 100 | 39.5 |
| 2 | 88 | 58.0 |
| 3 | 80 | 72.0 |
| 4 | 72 | 13.7 |
| 5 | 72 | 27.3 |
| 6 | 67 | 17.2 |
| 7 | 62 | 152.5 |
| 8 | 57 | 44.8 |
| 9 | 57 | 51.3 |
| 10 | 55 | 110.2 |
| 11 | 55 | 135.4 |
| 12 | 50 | 130.6 |
| 13 | 48 | 54.6 |
| 14 | 47 | 47.3 |
| 15 | 44 | 35.3 |
| 16 | 44 | 110.6 |
| 17 | 43 | 157.6 |
| 18 | 43 | 22.5 |
| 19 | 40 | 116.5 |
| 20 | 36 | 167.0 |
| 21 | 35 | 132.0 |
| 22 | 29 | 161.5 |
| 23 | 29 | 128.6 |

### EXAMPLE 3

### Crystalline Hydrate of Isomer M

### Part A: Preparation

Crystalline ethanolate of Isomer M (10 g) prepared in accordance with the method described in Part A of Example 2 was slurried in 100 mL of water at 30° C. and then aged at 30° C. with stirring for 24 hours. The crystalline solids were then separated from the slurry by filtration, washed with water (30 mL), and dried in an oven at 40° C. for 12 hours to provide the title product, which was determined to be a crystalline hydrate by the characterization studies described below.

### Part B: Characterization

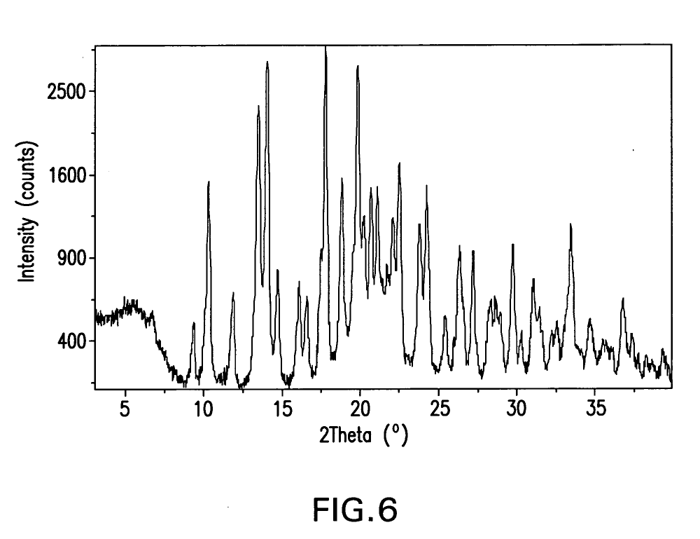

An XRPD pattern of crystalline hydrate prepared in accordance with the method described in Part A was generated using the same diffractometer and the same conditions as set forth in Part B of Example 2. The XRPD pattern is shown in FIG. 6. 2Θ values, the corresponding d-spacings, and the relative peak intensities in the XRPD pattern include the following:

TABLE 4
XRPD of crystalline hydrate

| Peak No. | d-spacing (Å) | 2 Theta | I/Imax (%) |
|---|---|---|---|
| 1 | 2.7 | 33.5 | 32 |
| 2 | 2.9 | 31.1 | 18 |
| 3 | 3.0 | 29.8 | 27 |
| 4 | 3.3 | 27.2 | 24 |
| 5 | 3.4 | 26.4 | 25 |
| 6 | 3.7 | 24.3 | 45 |
| 7 | 4.5 | 19.9 | 93 |
| 8 | 4.7 | 18.8 | 48 |
| 9 | 5.0 | 17.8 | 100 |
| 10 | 5.1 | 17.4 | 22 |
| 11 | 5.4 | 16.6 | 15 |
| 12 | 5.5 | 16.1 | 18 |
| 13 | 6.0 | 14.7 | 21 |
| 14 | 6.3 | 14.1 | 95 |
| 15 | 6.6 | 13.5 | 73 |
| 16 | 7.5 | 11.9 | 15 |
| 17 | 8.6 | 10.3 | 46 |

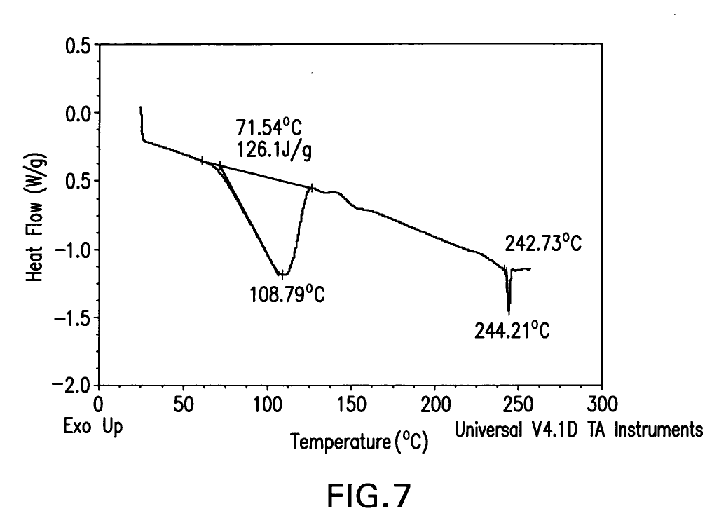

Crystalline hydrate prepared in accordance with the method described in Part A was also analyzed using the same differential scanning calorimeter described in Part A of Example 2 at a heating rate of 10° C./minute from 25° C. to 2650° C. in an open aluminum pan in a nitrogen atmosphere. The DSC curve (see FIG. 7) exhibited (i) a broad first endotherm with an onset temperature of 71° C., a peak temperature of 109° C., and an enthalpy change of 126 J/g and (ii) a narrow second endotherm with an onset temperature of 243° C. and a peak temperature of 244° C. The first endotherm is believed to be due to dehydration, and the second endotherm is believed to be due to melting.

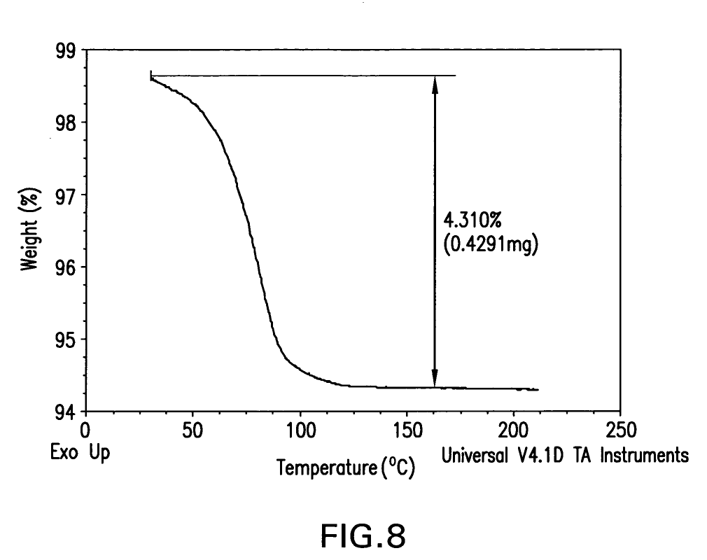

A thermogravimetric analysis of crystalline hydrate prepared in accordance with the method described in Part A was performed with TA Instruments TGA model Q 500 under nitrogen at a heating rate of 10° C./minute from 25° C. to 225° C. The TG curve (see FIG. 8) showed a weight loss of 4.3 wt. % up to 160° C.

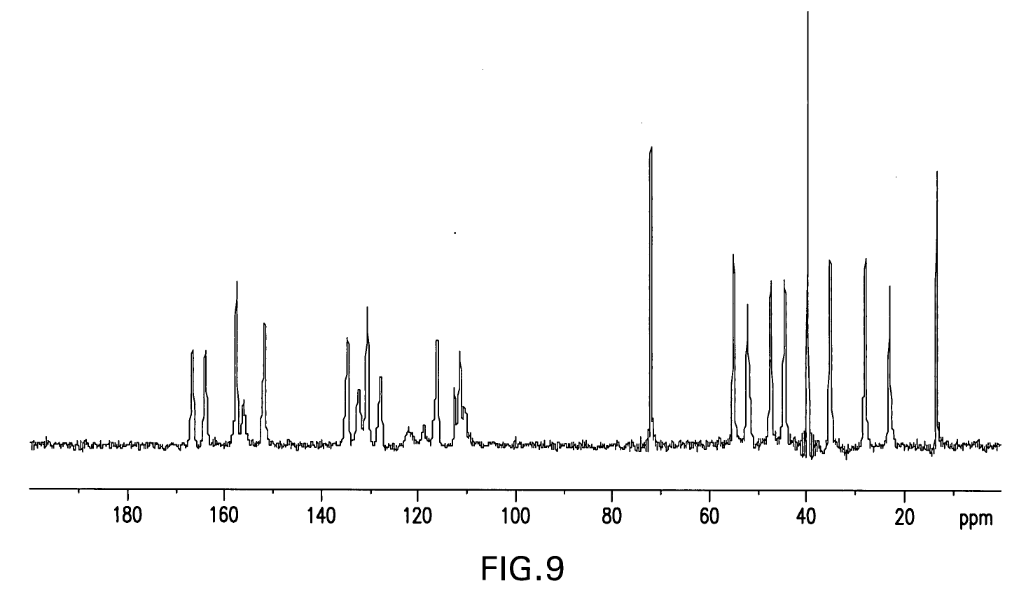

The crystalline hydrate was also characterized by solid-state carbon-13 NMR spectroscopy using the same instrument and conditions as described in Example 2. The spectrum is shown in FIG. 9, and chemical shift values are presented in the following table:

TABLE 5
Chemical shifts in the solid-state carbon-13 CPMAS NMR
spectrum of the crystalline hydrate

| | | Chemical Shift |
|---|---|---|
| Peak No. | I/Imax (%) | (ppm) |
| 1 | 100 | 39.8 |
| 2 | 69 | 72.1 |
| 3 | 63 | 13.3 |
| 4 | 44 | 55.0 |
| 5 | 43 | 28.0 |
| 6 | 43 | 35.1 |
| 7 | 38 | 47.3 |
| 8 | 38 | 44.5 |
| 9 | 38 | 157.6 |
| 10 | 37 | 23.0 |
| 11 | 33 | 52.2 |
| 12 | 32 | 130.6 |
| 13 | 28 | 151.7 |
| 14 | 25 | 134.6 |
| 15 | 25 | 116.2 |
| 16 | 22 | 163.9 |
| 17 | 22 | 166.6 |
| 18 | 22 | 111.5 |
| 19 | 16 | 127.9 |

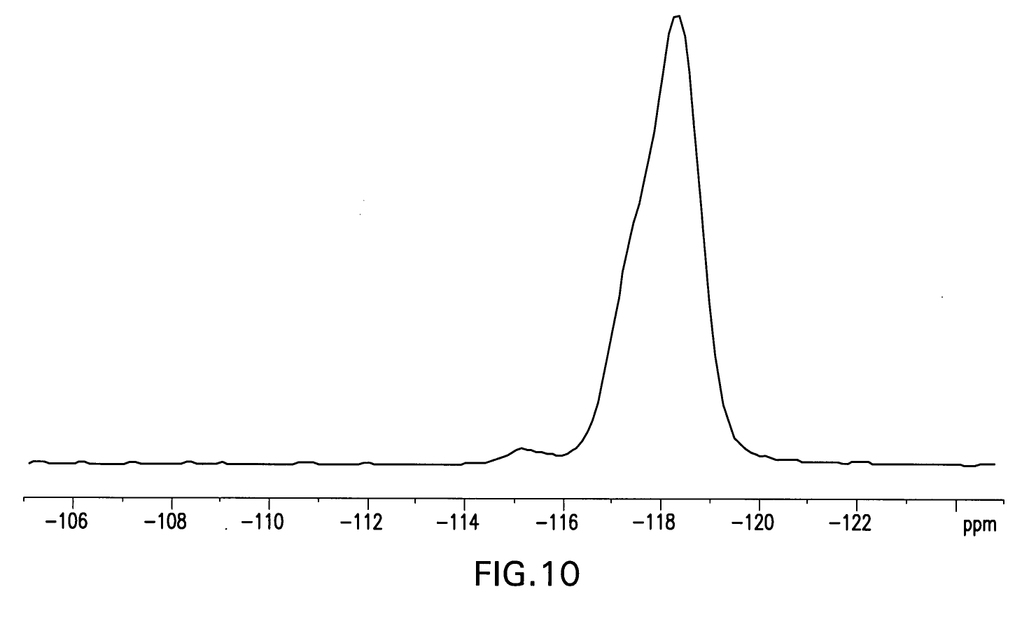

The hydrate form was further characterized by solid state fluorine-19 NMR. The solid-state fluorine-19 NMR spectra were obtained on a Bruker DSX 500WB NMR system using a Bruker 4 mm H/F/X CPMAS probe. The fluorine-19 NMR spectrum utilized proton/fluorine-19 cross-polarization magic-angle spinning with variable-amplitude cross polarization, and TPPM decoupling at 62.5 kHz. The sample was spun at 15.0 kHz, and a total of 256 scans were collected with a recycle delay of 5 seconds. A line broadening of 10 Hz was applied to the spectrum before FT was performed. The spectrum (see FIG. 10) contained a characteristic peak with a chemical shift of −118.4 ppm (reported using polytetrafluoroethylene (Teflon®) as an external secondary reference assigned a chemical shift of −122 ppm. A small peak in the spectrum was observed at −115.2 ppm and was attributed to a residual amount of crystalline anhydrate.

### EXAMPLE 4

### Crystalline Anhydrate of Isomer M

### Part A: Preparation

Crystalline ethanolate of Isomer M (10 g) prepared in accordance with the method described in Part A of Example 2 was slurried in 100 mL of MTBE at 25° C. and then aged at 25° C. with stirring for 15 hours. The crystalline solids were then separated from the slurry by filtration, washed with MTBE (30 mL), and dried in an oven at 40° C. for 12 hours to provide the title product, which was determined to be a crystalline anhydrate by the characterization studies described below.

### Part B: Characterization

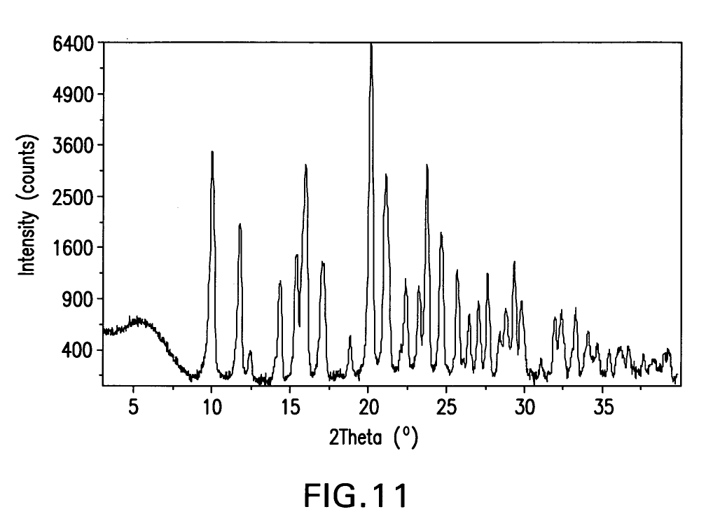

An XRPD pattern of crystalline anhydrate prepared in accordance with the method described in Part A was generated using the same diffractometer and the same conditions as set forth in Part B of Example 2. The XRPD pattern is shown in FIG. 11. 2Θ values, the corresponding d-spacings, and the relative peak intensities in the XRPD pattern include the following:

TABLE 6
XRPD of crystalline anhydrate

| Peak No. | d-spacing (Å) | 2 Theta | I/Imax (%) |
|---|---|---|---|
| 1 | 3.0 | 29.3 | 19 |
| 2 | 3.2 | 27.7 | 16 |
| 3 | 3.5 | 25.7 | 17 |
| 4 | 3.6 | 24.7 | 26 |
| 5 | 3.7 | 23.8 | 48 |
| 6 | 3.8 | 23.3 | 14 |
| 7 | 4.0 | 22.4 | 14 |
| 8 | 4.2 | 21.1 | 44 |
| 9 | 4.4 | 20.2 | 100 |
| 10 | 5.2 | 17.1 | 19 |
| 11 | 5.5 | 16.0 | 48 |
| 12 | 5.6 | 15.8 | 24 |
| 13 | 5.8 | 15.4 | 21 |
| 14 | 6.2 | 14.4 | 15 |
| 15 | 7.5 | 11.8 | 29 |
| 16 | 8.8 | 10.0 | 52 |

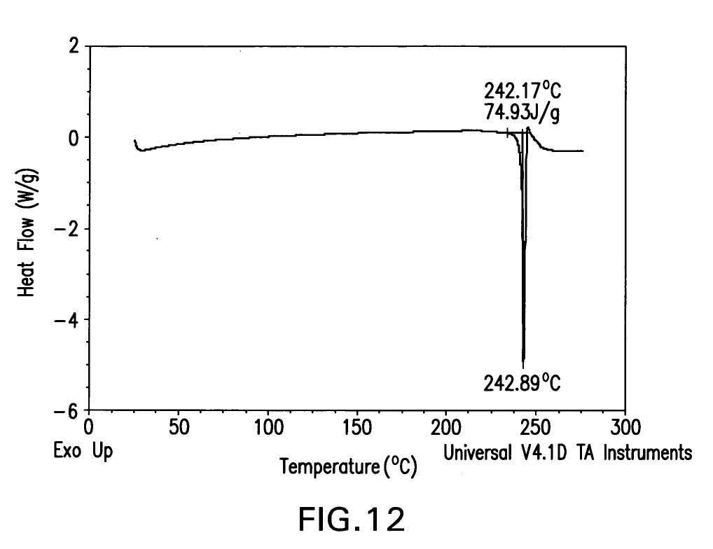

Crystalline anhydrate prepared in accordance with the method described in Part A was also analyzed using the same differential scanning calorimeter described in Part A of Example 2 at a heating rate of 10° C./minute from 25° C. to 260° C. in a covered aluminum pan in a nitrogen atmosphere. The DSC curve (see FIG. 12) exhibited an endotherm with an onset temperature of 242° C., a peak temperature of 243° C., and an enthalpy change of 75 J/g. The endotherm is believed to be due to melting.

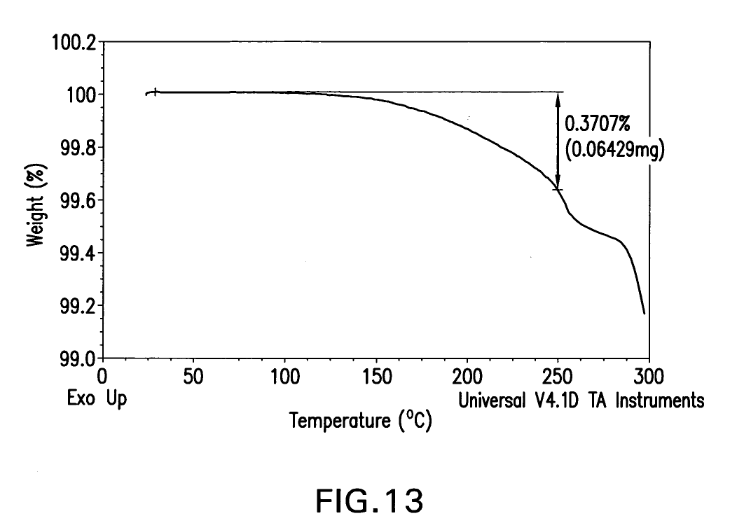

A thermogravimetric analysis of crystalline anhydrate prepared in accordance with the method described in Part A was performed with a TA Instruments TGA model Q 500 under nitrogen at a heating rate of 10° C./minute from 25° C. to 300° C. The TG curve (see FIG. 13) showed a weight loss of 0.4 wt. % up to 250° C.

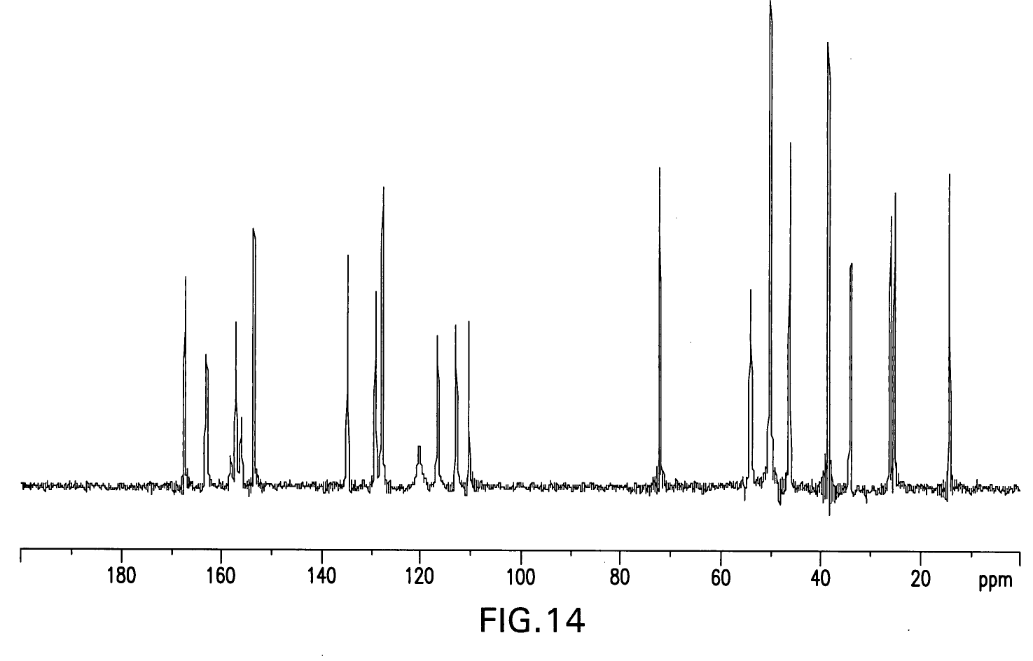

The crystalline anhydrate was also characterized by solid-state carbon-13 NMR spectroscopy using the same instrument and conditions as described in Example 3. The spectrum is shown in FIG. 14, and chemical shift values are presented in the following table:

TABLE 7
Chemical shifts in the solid-state carbon-13 CPMAS NMR
spectrum of the crystalline anhydrate

| | | Chemical Shift |
|---|---|---|
| Peak No. | I/Imax (%) | (ppm) |
| 1 | 100 | 50.1 |
| 2 | 92 | 38.5 |
| 3 | 71 | 46.2 |
| 4 | 66 | 72.2 |
| 5 | 65 | 14.3 |
| 6 | 62 | 127.8 |
| 7 | 61 | 25.3 |
| 8 | 57 | 26.1 |
| 9 | 53 | 153.6 |
| 10 | 48 | 134.8 |
| 11 | 47 | 34.0 |
| 12 | 43 | 167.3 |
| 13 | 41 | 129.2 |
| 14 | 40 | 54.0 |
| 15 | 35 | 110.4 |
| 16 | 34 | 113.0 |
| 17 | 34 | 157.1 |
| 18 | 31 | 116.7 |
| 19 | 27 | 163.0 |

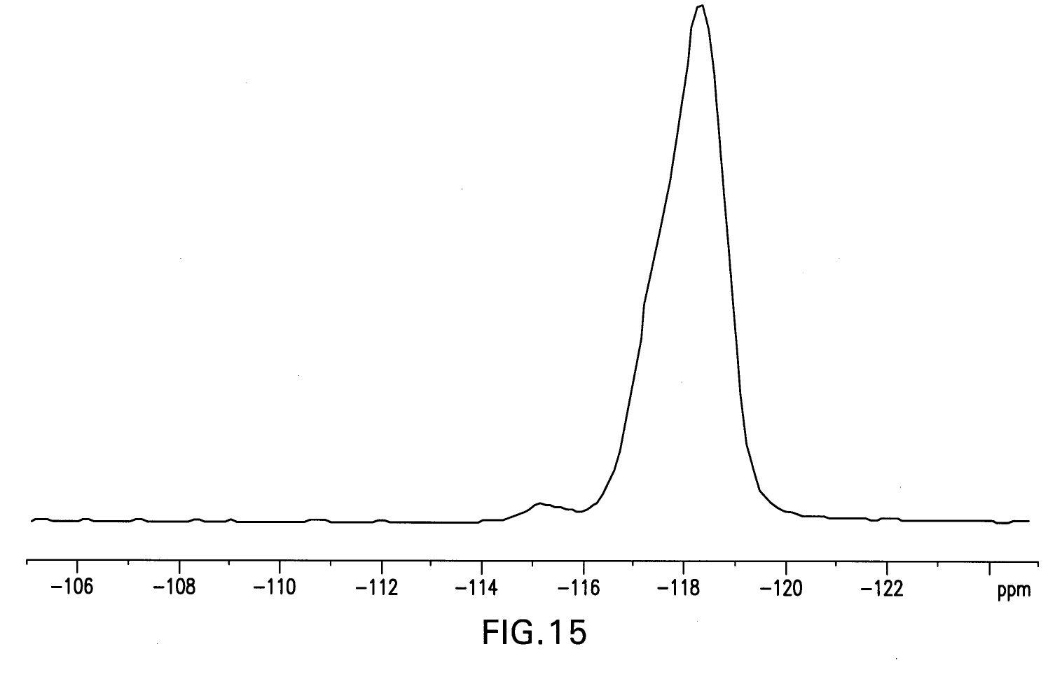

The anhydrate form was further characterized by solid state fluorine-19 NMR using the same instrument and conditions as described in Example 3. The spectrum (see FIG. 15) contained a characteristic peak with a chemical shift of −115.6 ppm.

### EXAMPLE 5

### Isomer M of (4R)-1-(3-chloro-4-fluorobenzyl)-4,9-dihydroxy-2,5,5-trimethyl-3,4,5,6,12,13-hexahydro-2H[1,4]diazocino[2,1-a]-2,6-naphthyridine-1,8,10(11H)-trione

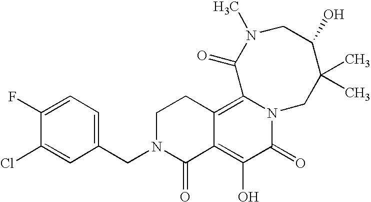

Step 1: Amino-[3-benzenesulfinyl-1-(3-chloro-4-fluoro-benzyl)-2-oxo-piperidin-4-yl]-acetic acid ethyl ester (5-1)

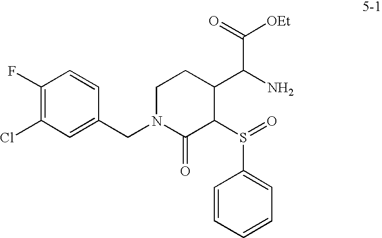

THF (40 L) was added to a 100 L flask, followed by the addition of phenyl 1-(3-chloro-4-fluorobenzyl)-2-oxo-5,6-dihydro-1(H)-pyridin-3-yl sulfoxide (5-0; 5.0 kg, 13.7 moles) and glycine diphenylamine (4.0 kg, 15.1 moles). The mixture was stirred at room temperature to dissolve the solids and the resulting solution was then cooled to 0° C. with an ice/water bath. Lithium t-butoxide (1 M in THF, 1.4 L, 1.4 moles) was then added dropwise while maintaining the temperature at less than 15° C. The mixture was aged at 0° C. for about one hour at which point complete conversion was achieved as determined by HPLC. Aqueous HCl (2 N, 35 L) was then added to the aged mixture at a rate that allowed the mixture to warm gradually to room temperature (approximately 15 minutes). The solution was then aged at room temperature for about 45 minutes at which point the hydrolysis of the imine intermediate was complete as determined by HPLC.

(HPLC Conditions: column=Waters Symmetry C-18 (250 mm×4.6 mm; 5 μm); flow rate=1.5 mL/minute; detection=210 nm; eluents=water 0.1% H₃PO₄ (A), acetonitrile (B); program=10% B 0 minute, 60% B 7 minutes, 80% B 18 minutes, 100% B 20 minutes; retention times=sulfoxide 5-0: 10.0 minutes, imine intermediate: 18.4 minutes, benzophenone: 12.0 minutes, saturated amino ester 5-1: 6.0 and 6.1 minutes (2 peaks)).

The solution was then charged to a 200 L extractor to which MTBE (25 L) was added. The resulting organic and aqueous layers were separated, and the organic layer was extracted with aqueous HCl (2 N, 5 L). The combined aqueous layers were washed with MTBE (2×25 L) to remove residual benzophenone. The acidic aqueous layer was recharged to a 100 L flask, along with IPAc (25 L) and the mixture cooled to 0° C. Aqueous NaOH (5 N, ˜25 L) was then added dropwise, while maintaining the temperature at less than 5° C., until the pH was 8.5. The layers were then separated, the aqueous layer re-extracted with IPAc (8 L), and the organic layers were combined to provide a solution of title compound 5-1 in IPAc. ES MS M+1=467.1.

Step 2: Amino-[1-(3-chloro-4-fluorobenzyl)-2-oxo-piperidin-(4Z)-ylidene]-acetic acid ethyl ester (5-2)

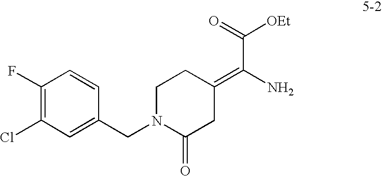

A solution of amino-[3-benzenesulfinyl-1-(3-chloro-4-fluoro-benzyl)-2-oxo-piperidin-4-yl]-acetic acid ethyl ester (5-1; 6.4 kg, 13.7 moles) in IPAc (see Step 1) was added to a 100 L flask. The solution was solvent switched to toluene and then adjusted to a total volume of 65 L (KF=200 ppm). Diisopropylethyl amine (2.4 L, 13.8 moles) was added to the resulting slurry, the flask was equipped with a water-cooled condenser, and the slurry heated to 90° C. to give a clear solution. The reaction mixture was determined by HPLC to have undergone full conversion after 30 minutes at 90° C. (HPLC Conditions: column=Waters Symmetry C-18 (250 mm×4.6 mm; 5 μm); flow rate=1.5 mL/minute, detection=210 nm; eluents=water 0.1% H₃PO₄ (A), acetonitrile (B); program=10% B 0 minutes, 60% B 7 minutes, 80% B 18 minutes, 100% B 20 minutes; retention times=saturated amino ester 5-1: 6.0 and 6.1 minutes (2 peaks); enamino ester 5-2: 5.4 and 8.4 minutes (2 peaks). The reaction mixture was then cooled to about 70° C., the condenser was removed, and the mixture concentrated by evaporation to about 18 L during which time a slurry formed. IPAc (2 L) was then added to the slurry which was then slowly cooled to room temperature and then aged at room temperature until the supernatant concentration was less than 16 mg/mL. The slurry was filtered, rinsed with 5:1 heptane:IPAC (12 L), and dried overnight on the filter pot with vacuum and nitrogen sweep to give title compound 5-2. ES MS M+1=341.1.

Step 3: 6-(3-Chloro-4-fluorobenzyl)-4-hydroxy-3,5-dioxo-2,3,5,6,7,8-hexahydro-2,6-naphthyridine-1-carboxylic acid ethyl ester (5-3)

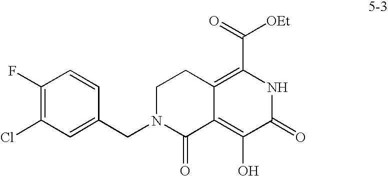

Imine 5-2 (3.50 kg, 8.80 moles) and THF (45 L) were charged to a 100 L flask, after which the mixture was cooled to about 0° C. and then charged with diisopropylethyl amine (1.70 L, 1.4 moles). Monoethyl oxalyl chloride (1.20 L, 9.24 moles) was added dropwise to the resulting solution at a rate such that the temperature was maintained below 3.5° C. The reaction mixture was then aged at 0° C. until the reaction was complete as determined by HPLC. (HPLC Conditions: column=Waters Symmetry C-18 (250 mm×4.6 mm; 5 μm); flow rate=1.5 mL/minute, detection=210 nm; eluents=water 0.1% H₃PO₄ (A), acetonitrile (B); program=10% B 0 minutes, 60% B 7 minutes, 80% B 18 minutes, 100% B 20 minutes; retention times=enamino ester 5-2: 5.4 and 8.4 minutes (2 peaks); intermediates: 9.4, 9.8, and 10.1 minutes.) Solid LiBr (3.06 kg, 35.2 moles) was then added to the aged mixture followed by the addition of DABCO (1.97 kg, 17.6 moles), after which the mixture was allowed to warm to room temperature and aged at room temperature overnight. The reaction mixture was then quenched with aqueous HCl (2 N, 35 L, 70 moles) and aged at room temperature for 30 minutes. Approximately 37 L of the original THF charge was then removed under reduced pressure, the resulting slurry diluted to the original volume with water, and the slurry aged at room temperature for 30 minutes and filtered. The wet cake was slurry washed with water (2×12 L), then with MTBE (3×12 L), and then dried under vacuum/N2 sweep until dry to provide title compound 5-3. ES MS M+1=395.0.

Step 4: 6-(3-Chloro-4-fluorobenzyl)-4-hydroxy-3,5-dioxo-2,3,5,6,7,8-hexahydro-2,6-naphthyridine-1-carboxylic acid (5-4)

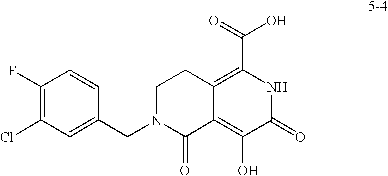

Ester 5-3 (3.08 kg, 7.8 moles) and a 1:1 mixture of EtOH:THF (37 L) were charged to a 100 L flask. Aqueous NaOH(SN, 46.8 moles, 9.4 L) was added to the resulting slurry and the slurry warmed to 50-53° C. for 45 minutes. The slurry was then diluted with water (10 L, ˜3.33 L/kg) and aged for an additional hour at 50-53° C., after which the batch was cooled to 15° C., made acidic by addition of concentrated HCl (72.6 moles, 6 L), and aged at room temperature for 10 hours. The slurry was then filtered, washed with water (3×12 L), and dried under vacuum/N2 sweep at 35° C. until dry to give title product 5-4 as a solid ES MS M+1=367.0.

Step 5: (R)-2,2-Dimethyl-4-methylamino-3-(tetrahydro-pyran-2-yloxy)-butan-1-ol (5)

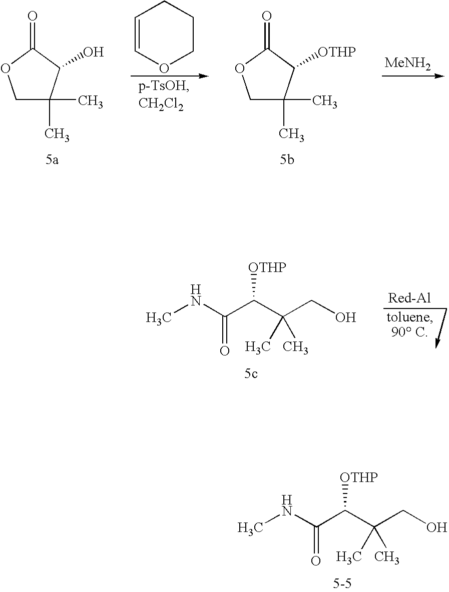

Dichloromethane (15.6 L), D-(−)-pantolactone (5.2 kg, 40.0 moles), and a catalytic amount of para-toluenesulfonic acid monohydrate (19.8 g, 0.1 moles) were charged to a 100 L flask, and the resulting solution was cooled to about −5° C. over a dry ice/acetone bath. 3,4-Dihydro-2H-pyran (4.0 L, 44.0 moles) was then added over 30 minutes while the temperature was kept below +20° C. The resulting solution was aged at room temperature for 1 hour, and then methylamine (33 wt %/EtOH, ca. 8 M, 12.4 L, 100.0 moles) was added in one portion. The resulting yellow solution was aged at room temperature for 20 hours. The reaction mixture was then diluted with toluene (15 L) and concentrated under vacuum to near dryness, and then flushed with toluene (20 L). The solution was then adjusted to a total volume of approximately 90 L (i.e., about 8 L toluene/kg of amide 5c). The toluene solution was then cooled to about −5° C. over a dry ice/acetone bath, and Red-Al (30.2 L, 100.0 moles) was added over approximately 45 minutes while maintaining the temperature at below +20° C. The resulting solution was heated to 90° C. for 4 hours, then allowed to cool to room temperature, then further cooled over a dry ice/acetone bath to about −5° C., and quenched by the slow addition (about 20 minutes) of IPA (8.7 L) while maintaining the temperature at below 20° C. The solution was then reverse added into a 20 wt. % aqueous KOH solution (52 L, 217.0 moles). The resulting layers were separated, and the organic layer washed with water (2×35 L) and concentrated to dryness to give title product 5-5 as an oil of diastereomeric mixture. ES-MS M+1=232.

Step 6: 6-(3-Chloro-4-fluorobenzyl)-N-[(2R)-4-hydroxy-3,3-dimethyl-2-(tetrahydro-2H-pyran-2-yloxy)butyl]-4-hydroxy-N-methyl-3,5-dioxo-2,3,5,6,7,8-hexahydro-2,6-naphthyridine-1-carboxamide (5-6)

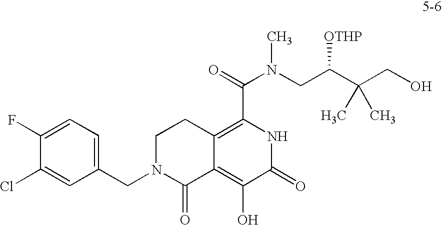

DMF (28.0 L), acid 5-4 (4.00 kg, 10.91 moles), aminoalcohol 5-5 (3.29 kg, 92 wt %, 13.09 moles), N-methylmorpholine (3.61 L, 32.73 moles), HOAt (74 g, 0.55 moles), and EDC (2.51 kg, 13.09 moles) were charged to a 75 L flask and the mixture stirred at room temperature for 60 hours. The reaction mixture was then diluted with ethyl acetate (40 L) and water (28 L), the resulting layers separated, and the aqueous layer back-extracted with EtOAc (40 L). The combined organic layers were washed with 2×40 L of water, and the resultant organic solution was azeotropically dried under reduced pressure with feeding of EtOAc to a final volume of approximately 50 L (KF<300 μg/g) to provide a solution of 5-6 in dry EtOAc. ES-MS M+1=581.2.

Step 7: Methanesulfonic acid (R)-4-{[6-(3-chloro-4-fluoro-benzyl)-3,4-bis-methanesulfonyloxy-5-oxo-5,6,7,8-tetrahydro-2,6-naphthyridine-1-carbonyl]-methyl-amino}-2,2-dimethyl-3-(tetrahydro-pyran-2-yloxy)-butyl ester (5-7)

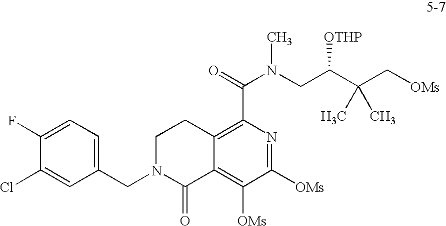

Triethylamine (6.08 L, 43.62 moles) was added to a solution of amide 5-6 (5.06 kg assay, 8.72 moles) in dry ethyl acetate and the solution cooled to +5° C. MsCl (3.38 L, 43.62 moles) was then slowly added while maintaining the temperature of the mixture below 20° C. The resulting slurry was aged at room temperature for one hour and then quenched to saturated aqueous NaHCO₃ solution (50 L). The biphasic solution was aged at room temperature for one hour and the layers then separated. The organic layer was washed with water (50 L), and the solvent then switched to DMAc under reduced pressure with a final volume of approximately 50 L (KF<300 μg/g) thereby providing title product 5-7 in DMAc solution. ES-MS M+1=815.1.

Step 8: Methanesulfonic acid (R)-4-{[6-(3-chloro-4-fluoro-benzyl)-3-methanesulfonyloxy-4-methoxy-5-oxo-5,6,7,8-tetrahydro-2,6-naphthyridine-1-carbonyl]-methyl-amino}-2,2-dimethyl-3-(tetrahydro-pyran-2-yloxy)-butyl ester (5-8)

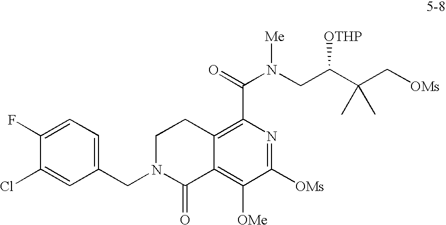

Cs₂CO₃ (5.40 kg, 16.58 moles) was added to a solution of the tris-mesylate 5-7 (6.75 kg assay, 8.29 moles) in DMAc and the mixture heated to 80° C. for two hours, after which the mixture was cooled to room temperature and MeI (1.55 L, 24.88 moles) added. The mixture was then aged at room temperature for 15 hours, after which water (40 L) and EtOAc (50 L) were added, the resulting layers separated, the organic layer washed with water (40 L) and then azeotropically dried with EtOAc and solvent switched with DMAc under reduced pressure to provide a solution of title product 5-8 in DMAc with a final volume of approximately 15 L (KF<300 μg/g). ES-MS M+1=751.1.

Step 9: (4R)-11-(3-Chloro-4-fluorobenzyl)-9-methoxy-2,5,5-trimethyl-4-(tetrahydro-2H-pyran-2-yloxy)-3,4,5,6,12,13-hexahydro-2H[1,4]diazocino[2,1-a]-2,6-naphthyridine-1,8,10(1H)-trione (5-9)

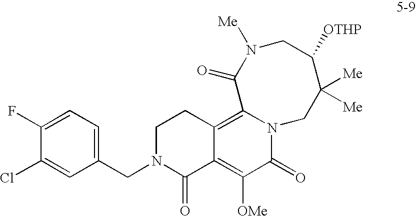

A solution of bis-mesylate 5-8 (5.66 kg assay, 7.54 moles) in DMAc (9 L) was slowly added over a 4-hour period to a 105° C. slurry of Cs₂CO₃ (7.37 kg, 22.63 moles) in DMAc (31 L), after which the mixture was aged at 105° C. for 3 hours and then cooled to room temperature. Water (50 L) was then added over a 1 hour period, and the slurry aged at room temperature for 15 hours. The slurry was then filtered, washed with 2×20 L of water, dried in the filter pot under vacuum/N2 sweep for 24 hours at room temperature to provide title product 5-9 (6.94 kg, ˜50 wt. %, 80% assay yield as determined by HPLC. ES-MS M+1=577.2.

HPLC Conditions: column=Waters Symmetry C-18 (250 mm×4.6 mm; 5 μm); flow rate=1.5 mL/minute, detection=210 nm; eluents=water 0.1% H₃PO₄ (A), acetonitrile (B); Program=20% B 0 minutes, 60% B 5 minutes, 90% B 15 minutes, 100% B 20 minutes; retention times=bis-mesylate 5-8: 9.31 minutes (broad); cyclized product 5-9: 8.09, 8.38, 8.74, 8.90 minutes (atropisomers+diastereomers).

Step 10: M-(R)-3-(3-Chloro-4-fluoro-benzyl)-9-hydroxy-5-methoxy-8,8,11-trimethyl-2,3,8,9,10,11-hexahydro-1H,7H-3,6a, 11-triaza-cycloocta[a]naphthalene-4,6,12-trione (5-10)

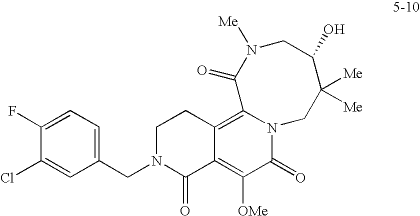

para-Toluenesulfonic acid monohydrate (0.343 kg, 1.81 moles) was added to a solution of THP ether 5-9 (3.47 kg assay, 6.02 moles) in methanol (30 L), and the mixture was heated to and maintained at 50° C. for 2 hours, after which the reaction mixture was allowed to cool to room temperature and was then solvent switched to ethyl acetate under reduced pressure with a final volume of 25 L (KF<1000 ppm) providing thereby a crystalline slurry containing title product 5-10 containing an 85/15 mixture of atropisomers M/P. The slurry was aged for two hours at room temperature, filtered, and washed with ETOAc (2×5 L). The cake was dried on the filter pot under vacuum/N2 sweep for 24 hours at room temperature to give 1.66 kg of title product 5-10 as the M-atropisomer (56% isolated yield). ES-MS M+1=492.1.

HPLC Conditions: column=Waters Symmetry C-18 (250 mm×4.6 mm; 5 μm); flow rate=1.5 mL/minute, detection=210 nm; eluents=water 0.1% H₃PO₄ (A), acetonitrile (B); Program=20% B 0 minutes, 60% B 5 minutes, 90% B 15 minutes, 100% B 20 minutes; retention times=THP ether 5-9: 8.09, 8.38, 8.74, 8.90 minutes (THP diastereomers+atropisomers); alcohol 5-10: 5.80 minutes (M-atropisomer), 5.91 minutes (P-atropisomer).

Step 11: M-(4R)-11-(3-chloro-4-fluorobenzyl)-4,9-dihydroxy-2,5,5-trimethyl-3,4,5,6,12,13-hexahydro-2H[1,4]diazocino[2,1-a]-2,6-naphthyridine-1,8,10(11H)-trione ethanolate (crystalline ethanolate of Isomer M) (5-11)

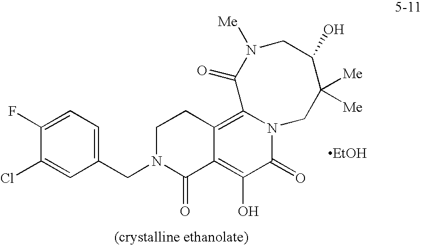

Boron tribromide (1M in CH₂Cl₂, 7.1 L, 7.1 moles) was slowly added to a solution of the methoxy ether 5-10 (1.66 kg, 3.37 moles) in CH₂Cl₂ (15 L) at 5° C. over 1 hour while maintaining the temperature below 20° C. Upon completion of the addition, the slurry was aged at room temperature for one hour with stirring and then cooled back to 5° C. Methanol (2.5 L) was then added slowly followed by the addition of water (20 L) in one portion, after which the mixture was aged at room temperature for one hour and the layers separated. The organic layer was washed with 1% aqueous NaHCO₃ (20 L) and then with water (20 L). The solvent was then switched to ethanol under reduced pressure to a final volume of approximately 16 L which led to the formation of a crystalline slurry which was aged at room temperature for two hours, filtered, and washed with EtOH (5 L). The cake was dried on the filter pot under vacuum/N2 sweep for 24 hours at room temperature to give 1.62 kg of the title crystalline ethanolate product as a pure atropisomer (<0.2 A % impurity) (i.e., crystalline ethanolate of Isomer M) in 92% isolated yield. ES-MS M+1=478.1. The crystalline form was confined by XRPD.

HPLC Conditions: column=Waters Symmetry C-18 (250 mm×4.6 mm; 5 μm); flow rate=1.5 mL/minute, detection=210 nm; eluants=water 0.1% H₃PO₄ (A), acetonitrile (B); Program=20% B 0 minutes, 60% B 5 minutes, 90% B 15 minutes, 100% B 20 minutes; retention times=methoxy ether 5.80 minutes (M-atropisomer), 5.91 minutes (P-atropisomer); crystalline ethanolate 5-11: 6.39 minutes (M-atropisomer), 6.49 minutes (P-atropisomer).

Step 12: M-(4R)-11-(3-chloro-4-fluorobenzyl)-4,9-dihydroxy-2,5,5-trimethyl-3,4,5,6,12,13-hexahydro-2H[1,4]diazocino[2,1-a]-2,6-naphthyridine-1,8,10(11H)-trione anhydrate (5-12) (crystalline anhydrate of Isomer M)

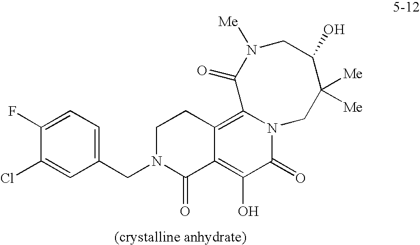

Crystalline ethanolate 5-11 (2.8 kg, 5.344 moles) was slurried in MTBE (28 L) with stirring at room temperature for 20 hours. The slurry was then filtered and washed with MTBE (5 L), and the filter dried on the filter pot under vacuum/N2 sweep at room temperature for 24 hours to give the title crystalline anhydrate of Isomer M (2.50 kg, 98% isolated yield). ES-MS M+1=478.1. The crystalline form was confirmed by XRPD. The HPLC conditions are the same as set forth in Step 11.

### EXAMPLE 6

### Solubility in Water

The solubilities of crystalline ethanolate, crystalline hydrate and crystalline anhydrate were determined in water at 25° C. using a ReactArray ST™ system (Anachem, UK). Approximately 50 mg of material was transferred to a sample tube and 1.5 mL of water was added to it. The tubes were mounted on a temperature control block and the slurry was stirred at 400 rpm using a magnetic stir bar. The solution was equilibrated for 24 hours following which the concentration was monitored every hour for 6 hours. The table below shows the average solubility measured during the last four hours.

HPLC conditions: column=C18 Bridge column (Waters Corporation), 4.6 mm×50 mm and 2.5 μm particle size; temperature=25° C., UV detection at λ=220 nm; flow rate=1 mL/minute; injection volume=5 μL; mobile phase=initial conditions at 20% acetonitrile and 80% 0.1% phosphate buffer with a gradient from 20% acetonitrile to 95% acetonitrile in 3 minutes, hold at 95% acetonitrile for 3 minutes, then from 95% to 20% acetonitrile in 0.1 minute (run time: 7 minutes), followed by 1 minute conditioning.

Water Solubility

| | Solubility | |
|---|---|---|
| Solid Form | (mg/mL) | Comment |
| Amorphous | 0.83 | Excess solid is amorphous |
| Crystalline ethanolate | 0.071 (±0.002) | Excess solid converts to hydrate |
| Crystalline hydrate | 0.068 (±0.002) | Excess solid is hydrate |
| Crystalline anhydrate | 0.124 (±0.010) | Excess solid is anhydrate |

*The results for the crystalline forms are based on 4 samples each and is reported as the mean ± SE. The result reported for the amorphous form is for 1 sample.

The results show that the solubility of the anhydrate form is higher than that of the other crystalline forms. The solubilities of the ethanolate and hydrate are nearly the same, which is consistent with the observed conversion of excess solid ethanolate to the hydrate during the course of the experiment. The amorphous material was significantly more soluble than any of the crystalline forms.

### Thermal Stability Studies

One set of samples of the amorphous, crystalline ethanolate, crystalline hydrate, and crystalline anhydrate forms of Isomer M were stored in a GMP stability chamber at 40° C. and 75% relative humidity (RH) and another set of analogous samples was stored at 60° C. and ambient RH in a Vacuum Oven, NAPCO (National Appliance Company) Model 5831. The samples were analyzed by XRPD and by HPLC before and after 2 weeks storage. The results are reported in the table below.

HPLC conditions: column=Symmetry Shield RP18 15 cm×4.6 mm (Waters Corporation) with 3.5 μm particle size; temperature=25° C., UV detection at λ=220 nm; flow rate=1 mL/minute; injection volume=10 μL; mobile phase=initial conditions at 20% acetonitrile and 80% 0.1% phosphate buffer with a gradient from 20% acetonitrile to 35% acetonitrile in 8 minutes, hold at 35% acetonitrile for 5 minutes, from 35% to 60% acetonitrile in 13 minutes, from 60% to 90% acetonitrile in 10 minutes (run time: 36 minutes, equilibration time: 10 minutes).

Thermal stability studies

| | Loss at 40° C., | Loss at 60° C., | |
|---|---|---|---|
| | 75% RH | ambient RH | |
| Form | (A %) | (A %) | XRPD |
| amorphous | 8.9 | not performed | not performed |
| crystalline | 0.3 | 11.5 | partial conversion to |
| ethanolate | | | anhydrate at 40° C., 75% |
| | | | RH |
| | | | no detectable change |
| | | | at 60° C., ambient RH |
| crystalline | 0.4 | 15.7 | no detectable change |
| hydrate | | | at 40° C., 75% RH |
| | | | partial conversion to |
| | | | amorphous form at |
| | | | 60° C., ambient RH |
| crystalline | NLQ | NLQ | no detectable change |
| anhydrate | | | in either case |

NLQ = no loss greater than the limit of quantitation

These results indicate that the crystalline anhydrate is the most stable of the forms of Isomer M and that each of the crystalline forms is significantly more stable than the amorphous form.

### EXAMPLE 8

### In Vivo Rat Pharmacokinetic Studies

In a single dose study (600 mg/kg), jugular vein surgically cannulated male Sprague-Dawley rats (3-4 rats/group) weighing approximately 300-350 grams each were given via oral gavage 2 mL/kg of 300 mg/g of either the crystalline ethanolate or crystalline anhydrate of Isomer M in suspensions in PEG 400. Blood samples were taken from all rats at pre-dose and at 0.25, 0.5, 1, 2, 4, 6, 8, and 24 hours post-dose.

Plasma levels of Compound A were quantified by LC-MS/MS analysis. The plasma samples were extracted using acetonitrile protein precipitation. HPLC analysis was carried out on a Shimadzu LC-10AD vp gradient system using the following parameters: column=Waters Atlantis dC18 (2.1 mm×50 mm, 5 μm); mobile phase=0.1% formic acid in water (solvent A) and 0.1% formic acid in acetonitrile (solvent B); flow rate=0.3 mL/minute; procedure=the initial solvent composition of 20% B was increased to 95% B over 1 minute, followed by holding solvent B constant at 95% for 2 minutes, and then returning to initial conditions for 1 minute. The HPLC system was interfaced with a PE Sciex API3000 mass spectrometer. Mass spectral analyses were carried out using a TurboIonSpray™ (TIS) source in the negative ionization mode. Quantitation was performed in multiple reaction monitoring (MRM) mode. The MRM transition used was m/z 476.016→291.900 for Compound A.

The Cₘₐₓ and AUC₀₋₂₄ hr values for the rats receiving the oral dose of crystalline ethanolate form of Isomer M were 46.5±7.8 μM and 624±90.1 μM-hr respectively (mean ±SE, n=3). The corresponding values for the anhydrate form were 43.5±4.89 μM and 755±94.4 μM·hr respectively (mean ±SE, n=4). Thus, the anhydrate and ethanolate forms were determined to have similar bioavailabilities. It is believed the anhydrate and ethanolate forms will have similar behavior in other animal models and in humans.

While the foregoing specification teaches the principles of the present invention, with examples provided for the purpose of illustration, the practice of the invention encompasses all of the usual variations, adaptations and/or modifications that come within the scope of the following claims.

What is claimed is:

1. A crystalline form of Isomer M, which is M-(4R)-11-(3-chloro-4-fluorobenzyl)-4,9-dihydroxy-2,5,5-trimethyl-3,4,5,6,12,13-hexahydro-2H[1,4]diazocino[2,1-a]-2,6-naphthyridine-1,8,10(11H)-trione.

2. A crystalline form according to claim 1, which is a crystalline ethanolate characterized by an X-ray powder diffraction pattern obtained using copper K_{α} radiation which comprises 2Θ values in degrees of about 17.8, 19.9, 21.0, and 21.8.

3. A crystalline ethanolate according to claim 2, which is further characterized by a carbon-13 CPMAS spectrum which comprises the chemical shifts in Table 3.

4. A crystalline form according to claim 1, which is a crystalline hydrate characterized by an X-ray powder diffraction pattern obtained using copper K_{α} radiation which comprises 2Θ values in degrees of about 13.5, 14.1, 17.8, and 19.9.

5. A crystalline hydrate according to claim 4, which is further characterized by a carbon-13 CPMAS spectrum which comprises the chemical shifts in Table 5.

6. A crystalline hydrate according to claim 4, which is further characterized by a fluorine-19 CPMAS spectrum having a chemical shift of about −118.4 ppm.

7. A crystalline form according to claim 1, which is a crystalline anhydrate characterized by an X-ray powder diffraction pattern obtained using copper K_{α} radiation which comprises 2Θ values in degrees of about 10.0, 16.0, 20.2, and 23.8.

8. A crystalline anhydrate according to claim 7, which is further characterized by a carbon-13 CPMAS spectrum which comprises the chemical shifts in Table 7.

9. A crystalline anhydrate according to claim 7, which is further characterized by a fluorine-19 CPMAS spectrum having a chemical shift of about −115.6 ppm.

10. A pharmaceutical composition comprising an effective amount of a crystalline form of Isomer M as recited in claim 1 and a pharmaceutically acceptable carrier.

11. The pharmaceutical composition according to claim 10, wherein the crystalline form of Isomer M is a crystalline anhydrate characterized by an X-ray powder diffraction pattern obtained using copper K_{α} radiation which comprises 2Θ values in degrees of about 10.0, 16.0, 20.2, and 23.8.

12. A process for preparing a crystalline ethanolate of Isomer M according to claim 2, which comprises:
(A) dissolving Isomer M in methylene chloride;
(B) switching the solvent to ethanol to provide a slurry of the crystalline ethanolate;
(C) optionally ageing the slurry; and
(D) optionally isolating the crystalline ethanolate.

13. A process for preparing a crystalline hydrate of Isomer M, which comprises:
(A) adding a crystalline ethanolate of Isomer M as recited in claim 2 to water to provide a slurry;
(B) ageing the slurry from Step A to provide the crystalline hydrate; and
(C) optionally isolating the crystalline hydrate.

14. A process for preparing a crystalline anhydrate of Isomer M, which comprises:
(D) forming a slurry of a crystalline ethanolate of Isomer M as recited in claim 2 in a slurrying agent selected from the group consisting of di-C₁-C₆ alkyl ethers, C₄-C₆ cyclic ethers, C₁-C₆ alkyl acetates and di-C₁-C₆ alkyl ketones, and optionally ageing the slurry, to obtain the crystalline anhydrate; and
(E) optionally isolating the crystalline anhydrate.

15. The process according to claim 14, wherein:
the forming and optional ageing of the slurry in Step D are each conducted at a temperature in a range of from about 5° C. to about 30° C.;
the slurrying agent is selected from the group consisting of MTBE, THF, EtOAc, IPAc, and acetone; and
the crystalline ethanolate in Step D is employed in an amount in a range of from about 0.01 g/mL to about 0.2 g/mL of the slurrying agent.

16. The process according to claim 14, which further comprises:
(B) reacting the M-atropisomer of 5-10: 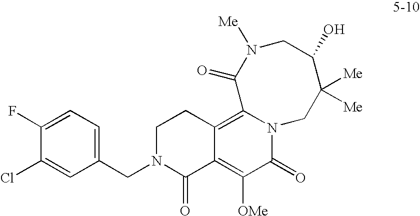
with boron tribromide to obtain Isomer M; and
(C) forming a slurry of Isomer M from Step B in ethanol, and optionally ageing the slurry, to obtain crystalline ethanolate of Isomer M.

17. The process according to claim 16, wherein:
the forming and optional ageing of the slurry in Step D are each conducted at a temperature in a range of from about 5° C. to about 30° C.;
the slurrying agent in Step D is selected from the group consisting of MTBE, THF, EtOAc, IPAc, and acetone;
the crystalline ethanolate in Step D is employed in an amount in a range of from about 0.01 g/mL to about 0.2 g/mL of the slurrying agent;
the reaction of Step B is conducted in methylene chloride at a temperature in a range of from about 5° C. to about 30° C.;
boron tribromide is employed in Step B in an amount in a range of from about 2.0 to about 4.0 equivalents per equivalent of 5-10; and
the forming and optional ageing of the slurry in step C are each conducted at a temperature in a range of from about 5° C. to about 30° C.; and
Isomer M in Step C is employed in an amount in a range of from about 0.01 g/mL to about 0.2 g/mL of ethanol.

18. The process according to claim 16, which further comprises:
(A) forming a slurry of a mixture of M- and P-atropisomers of 5-10 in a C₁-C₆ alkyl acetate, optionally ageing the slurry, and separating from the slurry M-atropisomer of 5-10.

19. The process according to claim 18, wherein:
the forming and optional ageing of the slurry in Step D are each conducted at a temperature in a range of from about 5° C. to about 30° C.;
the slurrying agent in Step D is selected from the group consisting of MTBE, THF, EtOAc, IPAc, and acetone;
the crystalline ethanolate in Step D is employed in an amount in a range of from about 0.01 g/mL to about 0.2 g/mL of the slurrying agent;
the reaction of Step B is conducted in methylene chloride at a temperature in a range of from about 5° C. to about 30° C.;
boron tribromide is employed in Step B in an amount in a range of from about 2.0 to about 4.0 equivalents per equivalent of 5-10;
the forming and optional ageing of the slurry in step C are each conducted at a temperature in a range of from about 5° C. to about 30° C.;
Isomer M in Step C is employed in an amount in a range of from about 0.01 g/mL to about 0.2 g/mL of ethanol;
the alkyl acetate in Step A is ethyl acetate or isopropyl acetate;
the forming and optional ageing of the slurry in Step A are each conducted at a temperature in a range of from about 5° C. to about 30° C.; and
the amount of 5-10 employed in the slurry in Step A is in a range of from about 5 g/mL to about 15 g/mL of alkyl acetate.
